(19) United States
(12) Patent Application Publication
(10) Pub. No.: US 2015/0342585 A1
(43) Pub. Date: Dec. 3, 2015

(54) SURGICAL INSTRUMENT

(71) Applicant: Agile EndoSurgery, Inc., Chapel Hill, NC (US)

(72) Inventor: Adam T.C. Steege, Chapel Hill, NC (US)

(21) Appl. No.: 14/821,088

(22) Filed: Aug. 7, 2015

Related U.S. Application Data
(63) Continuation of application No. 13/992,463, filed on Aug. 20, 2013.
(60) Provisional application No. 61/421,270, filed on Dec. 9, 2010.
(60) Provisional application No. 61/422,358, filed on Dec. 13, 2010.
(60) Provisional application No. 61/442,537, filed on Feb. 14, 2011.

(51) Int. Cl. A61B 17/00; A61B 17/32; A61B 17/3201; A61B 19/00

(52) U.S. Cl. CPC A61B 17/00234; A61B 19/22; A61B 17/320016; A61B 17/3201; A61B 2017/00327; A61B 2017/2902

(57) ABSTRACT

An end effector comprises a base portion defining a linear elongated slot extending along a longitudinal axis. A distal end of the base portion is adapted to be operatively mounted to an articulation mechanism. Each link of a first pair of links is pivotally connected to the base portion at a fixed pivot point. Each link of a second pair of links is pivotally connected at a fixed pivot point to one or the other of the first pair of links. The second pair of links is pivotally connected together through the slot of the base portion for linear movement along the slot relative to the base portion constraining movement of the first pair of links to relative rotation only in opposite directions.

### CROSS REFERENCE TO RELATED APPLICATIONS

This application is a continuation application of U.S. patent application Ser. No. 13/992,463 filed Aug. 20, 2013, which was filed under the provisions of 35 U.S.C. §371 and claims the priority of International Patent Application No. PCT/US2011/064086 filed Dec. 9, 2011, which claims the benefit of U.S. Provisional Application No. 61/421,270, filed Dec. 9, 2010, entitled “Surgical Tool Integrated Joint and End Effector,” U.S. Provisional Application No. 61/422,358, filed Dec. 13, 2010, entitled “Minimally Invasive Surgical Tool,” and U.S. Provisional Application No. 61/442,537, filed Feb. 14, 2011, entitled “Surgical Instrument,” the contents of all of which are hereby incorporated by reference in their entirety.

### FIELD

Embodiments described herein generally relate to surgical apparatus for tissue and suture manipulation, and more particularly may relate to apparatus that may be applied to conducting laparoscopic and endoscopic surgery.

### BACKGROUND

Minimally invasive surgery, such as endoscopic surgery, encompasses a set of techniques and tools which are becoming more and more commonplace in the modern operating room. Minimally invasive surgery causes less trauma to the patient when compared to the equivalent invasive procedure. Hospitalization time, scarring, and pain are also decreased, while recovery rate is increased.

Endoscopic surgery is accomplished by the insertion of a cannula containing a trocar to allow passage of endoscopic tools. Optics for imaging the interior of the patient, as well as fiber optics for illumination and an array of grasping and cutting devices are inserted through a multiple cannulae, each with its own port.

Currently the majority of cutting and grasping tools are essentially the same in their basic structure. Standard devices consist of a user interface at the proximal end and an end effector at the distal end of the tool used to manipulate tissue and sutures. Connecting these two ends is a tube section, containing cables and/or rods used for transmitting motion from the user interface at the proximal end of the tool to the end effector at the distal end of the tool. The standard minimally invasive devices (MIDs) provide limited freedom of movement to the surgeon. The cannula has some flexibility of movement at the tissue wall, and the tool can rotate within the cannula, but tools cannot articulate within the patient's body, limiting their ability to reach around or behind organs or other large objects. Several manually operated devices have attempted to solve this problem with articulated surgical tools that are controlled much in the same way as standard MIDs. These devices have convoluted interfaces, making them more difficult to control than their robotic counterparts. Many lack torsional rigidity, limiting their ability to manipulate sutures and denser tissue.

Robotic surgical instruments have attempted to solve the problems that arise from the limitations of standard MIDs with telemetrically controlled articulated surgical tools. However, these tools are often prohibitively expensive to purchase and operate. The complexity of the devices raises the cost of purchasing as well as the cost of a service contract. These robotic solutions also have several other disadvantages such as complications during the suturing process. An additional disadvantage can be difficulty in providing haptic feedback.

In the case of both articulated hand-held devices and robotic devices, the issue of compactness and strength are high priorities in terms of design. Many previously proposed articulated devices require a significant amount of space to articulate properly.

A newer form of MIS, known as Single Incision Laparoscopic Surgery (SILS) involves passing multiple tools through the same port. In order to avoid collisions between the interfaces of multiple systems, tools intended for SILS can be of varying lengths or be curved outside the patient's body. Even with these solutions to the issue of exterior instrument collisions, the instruments enter the abdomen from the same direction and are may be limited in their ability to manipulate tissue within the patient. Current articulated instruments may not have the capability to have their interfaces moved farther apart to prevent instrument collision exterior to the patient.

### SUMMARY

In accordance with one embodiment, a surgical instrument for use by an operator is provided. The surgical instrument includes a manipulator adapted to receive at least a portion of the operator's hand. A proximal universal joint has a first end and a second end, with the first end being mounted to the manipulator. A hollow elongated member has a first end, a second end, and a longitudinal axis, with the elongated member first end being mounted to the proximal universal joint second end. A distal universal joint has a first end and a second end, with the distal universal joint first end being mounted to the elongated member second end. An end effector includes at least one movable jaw, and is mounted to the distal universal joint second end. Cables operatively couple the manipulator, proximal universal joint, and distal universal joints and concurrently operatively couple the manipulator and the end effector.

In some embodiments, the cables include four cable lengths that control two degrees of freedom of the distal universal joint and one degree of freedom of the at least one movable jaw. The four cable lengths may include, for example, two cables terminating in the manipulator and fixed to the end effector, or four separate cables, each terminating in the manipulator and in the end effector.

In some embodiments, the manipulator includes a tensioning assembly with an anchor to which an end of each cable is attached. Pivoting of the first end of the proximal universal joint causes the second end of the distal universal joint to move in a corresponding pivoting motion, and actuation of the anchor operates the at least one movable jaw.

In some embodiments, the cables comprise four cable lengths that control both the pivoting of the second end of the distal universal joint and the operation of the at least one movable jaw. In some such embodiments, the at least one movable jaw comprises two movable jaws that operate simultaneously.

In some embodiments, the proximal universal joint and distal universal joint each include a proximal yoke at the first end, a distal yoke at the second end, and a center block between the proximal yoke and distal yoke. Means for mounting the proximal yoke and the distal yoke to the center block permit pivoting the proximal yoke and distal yoke about two perpendicular, coplanar axes through the respective center block.

In some such embodiments, each proximal yoke is mounted to the respective center block at first and second mounting locations and each distal yoke is mounted to the respective center block at third and fourth mounting locations. Between each center block and each yoke at each mounting location are round features, which may be independent parts or integral to either of the center block or yokes. Each of the four cable lengths engage two of the round features at each of the proximal and distal universal joints, pivoting the proximal yoke on the proximal universal joint causes a corresponding motion of the distal yoke of the distal universal joint.

In some embodiments, each center block is substantially cylindrical and comprises a round feature at each end. In some embodiments, the manipulator further comprises a housing to which the anchor is pivotally mounted, wherein actuation of the anchor results in retraction of at least one cable to result in movement of the at least one jaw. In some such embodiments, the manipulator further comprises first and second lever assemblies that move concurrently to actuate the anchor. In some embodiments, the tensioning assembly further comprises vented screws mounted to the anchor, and wherein the cables pass through the vented screws and are held in place. In some embodiments, the anchor includes a substantially u-shaped flange and a web across the flange, and the anchor pivots about a pin mounted to the housing. The vented screws are mounted to the flange. In some embodiments, a linkage between the first lever assembly and the anchor and between the second lever assembly and the anchor for each lever assembly is provided to apply force to pivot the anchor. In some embodiments, the first lever assembly is adapted to receive the index finger of a person's hand, and the second lever assembly is adapted to receive the thumb of the same hand.

In some embodiments, the manipulator comprises a brake that maintains the angular position of the manipulator relative to the elongated member. In some embodiments, a joint guard proximate to the proximal universal joint is provided. The joint guard has an inside that defines a substantially concave surface. The brake applies pressure to the inside concave surface to maintain the angular position of the manipulator relative to the elongated member. In some embodiments, the manipulator further comprises a brake trigger configured to apply the brake. In some embodiments, a brake is provided that maintains the angular position of the manipulator relative to the elongated member, and the manipulator further comprises a brake trigger configured to apply the brake. In some embodiments, the manipulator includes a brake trigger lock to maintain the brake trigger in position when the brake is applied.

In some embodiments, the manipulator comprises a handlebar and a handlebar lock that may be released to switch the handlebar between a first mounting position for engagement of the handlebar by a person's right hand and a second mounting position for engagement of the handlebar by a person's left hand. In some embodiments, the manipulator includes a pistol-grip handle portion.

In some embodiments, the elongated hollow member includes a first rigid section with a proximal end mounted to the proximal joint and a distal end, a middle section with a proximal end mounted to a distal end of the first rigid section and a distal end, and a second rigid section with a proximal end mounted to the distal end of the middle section and a distal end mounted to the distal joint. In some such embodiments, the middle section permits the first rigid section and the second rigid section to be offset from one another, and a locking mechanism is provided for securing the relative positions of the first rigid section and the second rigid section. In some such embodiments, the middle section includes a flexible material. In other such embodiments, the middle section is rigid and is mounted to the first and second rigid sections with universal joints.

In some embodiments, the proximal universal joint and distal universal joint each include a proximal end member and a distal end member, with each end member including a base portion and opposing arms extending from the base portion. The arms of each proximal end member and each distal end member are mounted to a respective center block for each joint at mounting locations. The center block defines with the mounting locations two substantially coplanar, perpendicular axes about which the proximal end member of the proximal universal joint and the distal end member of the distal universal joint may pivot.

In accordance with another embodiment, another surgical instrument for use by an operator is provided. A manipulator is adapted to receive at least a portion of the operator's hand. A proximal universal joint has a first end and a second end, with the proximal universal joint first end being mounted to the manipulator. A hollow elongated member has a first end, a second end, and a longitudinal axis, with the elongated member first end being mounted to the proximal universal joint second end. An end segment includes an integrated distal universal joint and end effector. The end segment has a first end mounted to the elongated member second end and a second end, and includes at least one movable jaw. Cables are provided that operatively couple the manipulator, proximal universal joint, and distal universal joints and that concurrently operatively couple the manipulator and the at least one movable jaw.

In some embodiments, the cables comprise four cable lengths that control three degrees of freedom of the end segment. In some such embodiments, the four cable lengths may include, for example, two cables terminating in the manipulator and fixed to the end segment, or four separate cables, each terminating in the manipulator and in the end segment.

In some embodiments, the manipulator includes a tensioning assembly with an anchor to which an end of each cable is attached. Pivoting of the first end of the proximal universal joint causes the second end of the end segment to move in a corresponding pivoting motion, and actuation of the anchor operates the at least one movable jaw.

In some embodiments, the cables comprise four cable lengths that control both the pivoting of the second end of the end segment and the operation of the at least one movable jaw.

In some embodiments, the proximal universal joint includes a first proximal yoke at the first end of the proximal universal joint, a first distal yoke at the second end of the proximal universal joint, and a first center block between the proximal yoke and distal yoke of the proximal universal joint. Means for mounting the proximal yoke and the jaw base to the first center block permit pivoting the proximal yoke and distal yoke about two perpendicular, coplanar axes through the first center block. The end segment include a second proximal yoke at the first end of the end segment, a jaw base including a distal yoke portion and a fixed jaw at the second end of the end segment, and a second center block between the second proximal yoke and the jaw base. Means for mounting the proximal yoke and the jaw base to the second center block permit pivoting the proximal yoke and distal yoke about two perpendicular, coplanar axes through the second center block.

In some such embodiments, each proximal yoke is mounted to the respective center block at first and second mounting locations, and the first distal yoke and the distal yoke portion are mounted to the respective center block at third and fourth mounting locations. Between each center block and each yoke and the distal yoke portion at each mounting location are round features. The round features may be independent parts or integral to either of the center block or yokes or distal yoke portion. Each of the four cable lengths engage two of the round features at each of the proximal universal joint and the end segment, and pivoting the proximal yoke on the proximal universal joint causes a corresponding motion of the distal yoke portion of the end segment.

In some embodiments, the proximal universal joint includes a first proximal end member and a first distal end member, with each end member including a base portion and opposing arms extending from the base portion. The arms of the first proximal end member and the first distal end member are mounted to a first center block at mounting locations. The first center block defines with the mounting locations two substantially coplanar, perpendicular axes about which the first proximal end member of the proximal universal joint may pivot. The end segment includes a second proximal end member and a jaw base, with the second proximal end member including a base portion and opposing arms extending from the base portion. The jaw base includes a base portion and opposing arms extending from the base portion, a body, a fixed jaw extending from the body, a point of mounting for a moveable jaw, and opposing arms extending from the body. The arms of the second proximal end member and the jaw base are mounted to a second center block at mounting locations, with the second center block defining with the mounting locations two substantially coplanar, perpendicular axes about which the jaw base may pivot.

In accordance with another embodiment, a manipulator for a surgical instrument to be operated by a user is provided. The surgical instrument includes cable lengths operatively coupling a proximal joint and a distal joint, with an elongated hollow member between the joints. An end effector is mounted to the distal joint and includes at least one movable jaw. The manipulator includes a housing, a handle portion operatively connected to the housing, and a member extending from the housing and configured to be operatively connected to the proximal joint. An anchor is pivotally mounted to the housing, and is configured to receive and secure an end of each of the cables lengths such that pivoting the anchor retracts at least one cable length into the housing and operates the at least one movable jaw. A mechanism is provided that is configured to receive force input by the user for actuating the anchor.

In some embodiments, a first lever assembly configured for receiving a user's index finger and a second lever assembly configured for receiving the user's thumb are provided. The first lever assembly and the second lever assembly are pivotally mounted to the housing for actuating the anchor. In some embodiments, a jaw trigger pivotally mounted to the handle portion for actuating the anchor is provided. In some such embodiments, a jaw trigger lock is provided for maintaining the jaw trigger in an actuated position. In some embodiments, a brake is provided that is configured to secure the manipulator in a selected angular position with respect to the elongated hollow member. In some such embodiments, a brake trigger configured to actuate the brake.

In some embodiments, the handle portion is configured as a handlebar, and further comprising a base member to which the handlebar is pivotally mounted. The handlebar may include two handles, and the handlebar may be pivoted to be configured to receive the user's right hand in a first orientation or the user's left hand in a second orientation. In some such embodiments, a handlebar lock is provided to secure the handlebar at the base member in either the first orientation or the second orientation. In some embodiments, the handle portion is configured as a pistol-grip.

In accordance with another embodiment, another manipulator for a surgical instrument is provided to be operated by a user. The surgical instrument includes an end effector mounted to an elongated hollow member and including at least one movable jaw, with cable lengths fixed to the end effector. The manipulator includes a housing, a handle portion operatively connected to the housing, and a member extending from the housing and configured to be operatively connected to the elongated member. An anchor is pivotally mounted to the housing and is configured to receive and secure an end of each of the cable lengths such that pivoting the anchor retracts at least one cable length into the housing and operates the at least one movable jaw. A mechanism is provided that is configured to receive force input by the user for actuating the anchor.

In accordance with another embodiment, an end segment for a surgical instrument is provided. The end segment includes a proximal yoke at the first end of the end segment. A jaw base is provided including a distal yoke portion and a fixed first jaw at the second end of the end segment. A center block is provided between the proximal yoke and the jaw base. Means for mounting the proximal yoke and the jaw base to the center block permit pivoting the proximal yoke and distal yoke about two perpendicular, coplanar axes through the center block. A second jaw is pivotally mounted to the jaw base.

In accordance with another embodiment, an elongated hollow member for a surgical instrument is provided. The elongated hollow member is configured to allow cables to pass therethrough for operating an end effector of the surgical instrument. The elongated hollow member includes a first rigid section with a proximal end and a distal end, a middle section with a proximal end mounted to a distal end of the first rigid section and a distal end, and a second rigid section with a proximal end mounted to the distal end of the middle section and a distal end mounted to the distal joint. In some embodiments, the middle section permits the first rigid section and the second rigid section to be offset from one another, and further comprising a locking mechanism for securing the relative positions of the first rigid section and the second rigid section. In some such embodiments, the middle section includes a flexible material, and in other such embodiments the middle section is rigid and is mounted to the first and second rigid sections with universal joints.

In accordance with another embodiment, a method of operating a surgical instrument is provided. The surgical instrument includes a manipulator adapted to receive at least a portion of the operator's hand and including a pivotally mounted anchor. A proximal universal joint has a first end and a second end, with the proximal universal joint first end being mounted to the manipulator. A hollow elongated member has a first end, a second end, and a longitudinal axis, with the elongated member first end being mounted to the proximal universal joint second end. A distal universal joint has a first end and a second end, with the distal universal joint first end being mounted to the elongated member second end. An end effector is mounted to the distal universal joint second end and includes at least one movable jaw. Cable lengths operatively couple the manipulator, proximal universal joint, and distal universal joints and concurrently operatively couple the manipulator and the end effector. The method includes pivoting the manipulator relative to the longitudinal axis of the elongated member to pivot the first end of the proximal universal joint. At least one cable length is retracted with the pivoting of the proximal universal joint to cause the second end of the distal universal joint to pivot. The anchor is actuated to retract at least one cable length to operate the at least one moveable jaw.

In accordance with another embodiment, another method of operating a surgical instrument is provided. The surgical instrument includes a manipulator adapted to receive at least a portion of the operator's hand and including a pivotally mounted anchor. A proximal universal joint has a first end and a second end, with the proximal universal joint first end being mounted to the manipulator. A hollow elongated member has a first end, a second end, and a longitudinal axis, with the elongated member first end being mounted to the proximal universal joint second end. An end segment including an integrated distal universal joint and end effector is provided, with the end segment having a first end, a second end, and at least one moveable jaw. The end segment first end is mounted to the elongated member second end. Cable lengths operatively couple the manipulator, proximal universal joint, and distal universal joints and concurrently operatively couple the manipulator and the end effector. The method includes pivoting the manipulator relative to the longitudinal axis of the elongated member to pivot the first end of the proximal universal joint. At least one cable length is retracted with the pivoting of the proximal universal joint to cause the second end of the end segment to pivot. The anchor is actuated to retract at least one cable length to operate the at least one moveable jaw.

Another embodiment of an end effector for a surgical instrument is provided. The end effector has a distal end including an articulation mechanism. The end effector comprises a base portion having a longitudinal axis, the base portion defining a linear elongated slot extending along the longitudinal axis. The base portion is adapted to be operatively mounted to the articulation mechanism. The end effector further comprises a first pair of links and a second pair of links. Each link of the first pair of links has a first end and a second end, the first ends of the first pair of links are pivotally connected to the base portion at a fixed pivot point. Each link of the second pair of links has a first end and a second end and is pivotally connected at a fixed pivot point to one or the other of the first pair of links. The second pair of links is pivotally connected together through the slot of the base portion for linear movement along the slot relative to the base portion. Linear movement of the pivotally connected second pair of links relative to the base portion constrains the movement of the first pair of links to relative rotation only in opposite directions.

Also provided is an embodiment of a surgical instrument comprising an articulation mechanism and an end effector. The end effector includes a base portion having a longitudinal axis, the base portion defining a linear elongated slot extending along the longitudinal axis. The base portion is configured to be operatively mounted to the articulation mechanism. Each link of a first pair of links has a first end and a second end. The first ends of the first pair of links are pivotally connected to the base portion at a fixed pivot point. Each link of a second pair of links has a first end and a second end and is pivotally connected at a fixed pivot point to one or the other of the first pair of links. The second pair of links is pivotally connected together through the slot of the base portion for linear movement along the slot relative to the base portion. The second pair of links is configured for concurrent relative rotation only in opposite directions.

Further features of a surgical instrument will become more readily apparent from the following detailed description taken in conjunction with the accompanying drawings.

### BRIEF DESCRIPTION OF THE DRAWINGS

For a more complete understanding, reference should now be had to the embodiments shown in the accompanying drawings and described below. In the drawings:

FIG. 1 is a right perspective view from above of a first embodiment of a surgical instrument;

FIG. 2 is a right perspective view from above of the surgical instrument of FIG. 1 in an articulated position.

FIG. 3 is a top plan view of the surgical instrument of FIG. 1 in an articulated position;

FIG. 4 is a left side view of the surgical instrument of FIG. 1 in an articulated position;

FIG. 5 is a right perspective view from above of the instrument in FIG. 3 in a non-articulated position with the end effector and manipulator in an open position;

FIG. 6 is an exploded view of the instrument of FIG. 1;

FIG. 7 is a right perspective view from above of an embodiment of an end effector and distal joint assembly as shown in the surgical instrument of FIG. 1;

FIG. 8 is an exploded view of the end effector and distal joint assembly of FIG. 7;

FIG. 9 is a right perspective view from above of one of the jaws of the end effector of FIG. 7 with cabling;

FIG. 10 is a left perspective view from above of the jaw and cabling of FIG. 9;

FIG. 11 is a first section perspective view of the right side of the end effector and distal joint assembly of FIG. 7;

FIG. 12 is a second section perspective view of the top of the end effector and distal joint assembly of FIG. 7;

FIG. 13 is a third section perspective view of the left side of the end effector and distal joint assembly of FIG. 7;

FIG. 14 is a fourth section perspective view of the bottom of the end effector and distal joint assembly of FIG. 7;

FIG. 15 is a right perspective view from above of the end effector and distal joint assembly in FIG. 7 with the jaws in an open position;

FIG. 16 is a fifth section view of the end effector and distal joint assembly of FIG. 7 in the position shown in FIG. 15;

FIG. 17 is a perspective view of an embodiment of an articulation system of the surgical instrument shown in FIG. 1, including embodiments of a proximal universal joint, distal universal joint, and end effector;

FIG. 18 is a right perspective view of a distal universal joint and an end effector of FIG. 7 articulated about a first axis.

FIG. 19 is a right perspective view of a distal universal joint and an end effector of FIG. 7 articulated about a second axis.

FIG. 20 is a right perspective view of another embodiment of an end effector, with the jaws in the closed position.

FIG. 21 is a right perspective view of the end effector of FIG. 20, with the jaws in the open position.

FIG. 22 is an exploded perspective view of the end effector of FIG. 20.

FIG. 23 is a right perspective view of an embodiment of the manipulator assembly of the surgical instrument of FIG. 1 in a right-handed configuration;

FIG. 24 is a section view of the manipulator assembly of FIG. 23;

FIG. 25 is a right perspective view of the manipulator assembly of FIG. 23 with the brake trigger released.

FIG. 26 is an exploded view of the manipulator assembly of FIG. 23;

FIG. 27 is a first section view of the manipulator assembly of FIG. 23 including the proximal joint and cabling;

FIG. 28 is a second section view of the manipulator assembly of FIG. 23 in an open position including the proximal joint and cabling;

FIG. 29 is a right perspective view from above of an embodiment of an index assembly from the manipulator assembly of FIG. 20;

FIG. 30 is an exploded view of the index assembly of FIG. 29;

FIG. 31 is a section view of the index assembly of FIG. 29;

FIG. 32 is a right perspective view from above of an embodiment of a handlebar assembly from the manipulator assembly of FIG. 23, in a neutral configuration;

FIG. 33 is an exploded view of the handlebar assembly of FIG. 32;

FIG. 34 is a first right section view from above of the handlebar assembly of FIG. 32;

FIG. 35 is a second right section view of the handlebar assembly of FIG. 32;

FIG. 36 is a right perspective view from below of the handlebar assembly of FIG. 32 in a right-handed configuration;

FIG. 37 is a section view of the handlebar assembly in the perspective of FIG. 36;

FIG. 38 is a right perspective view from below of the handlebar assembly in FIG. 36 with the trigger in a retracted position;

FIG. 39 is a right perspective view from above of the surgical instrument of FIG. 1 including a first alternate embodiment of a tube assembly;

FIG. 40 is a right perspective view from above of the instrument of FIG. 39 with the tube assembly in an offset configuration;

FIG. 41 is an exploded view of the tube assembly of FIG. 39;

FIG. 42 is a right section view of the tube assembly as shown in FIG. 39;

FIG. 43 is a right section view of the tube assembly as shown in FIG. 40;

FIG. 44 is a right perspective view from above of a second alternate embodiment of the tube assembly of FIG. 1;

FIG. 45 is a right perspective view from above of the tube assembly of FIG. 44 in an offset configuration;

FIG. 46 is an exploded view of the tube assembly of FIG. 44.

FIG. 47 is a right perspective view of a second embodiment of a surgical instrument.

FIG. 48 is an exploded view of the surgical instrument of FIG. 47.

FIG. 49 is a right perspective view from above of an embodiment of an end effector with an integrated distal joint as in the surgical instrument of FIG. 47;

FIG. 50 is a right section view from above of the end effector of FIG. 49;

FIG. 51 is an exploded view of the end effector of FIG. 49;

FIG. 52 is a left section view of the end effector of FIG. 49;

FIG. 53 is a right section view of the end effector of FIG. 49;

FIG. 54 is a right section view of the end effector of FIG. 49, showing the jaw articulated about a first joint axis;

FIG. 55 is a right section view of the end effector of FIG. 49, showing the jaw articulated about a second joint axis;

FIG. 56 is a right perspective view of an embodiment of the manipulator of the surgical instrument of FIG. 47.

FIG. 57 is a section view of the manipulator of FIG. 56.

FIG. 58 is a right front perspective view of the manipulator of FIG. 56.

FIG. 59 is an exploded view of the manipulator of FIG. 56.

FIG. 60 is a left perspective view from above of an embodiment of the control assembly in the manipulator of FIG. 56.

FIG. 61 is an exploded view of the control assembly of FIG. 60.

FIG. 62 is a right section view of the proximal portion of the instrument of FIG. 47 with the housing and trigger elements of the manipulator of FIG. 56 removed.

FIG. 63 is a left front perspective view of the jaw trigger of the manipulator of FIG. 56.

FIG. 64 is a left rear perspective view of the jaw trigger of FIG. 63.

FIG. 65 is a top perspective view of the brake trigger of FIG. 59.

FIG. 66 is a left side view of the brake trigger of FIG. 65.

FIG. 67 is a front view of the brake trigger of FIG. 65.

FIG. 68 is a left front perspective view of the brake actuating element of the control assembly of FIG. 60.

FIG. 69 is a left front perspective view of an embodiment of a jaw actuating element of the control assembly of FIG. 60.

FIG. 70 is a front view of the jaw actuating element of FIG. 69.

FIG. 71 is a left view of the jaw actuating element of FIG. 69.

FIG. 72 is a right perspective view of another embodiment of an end effector, with the jaws in the closed position.

FIG. 73 is a right perspective view of the end effector of FIG. 72, with the jaws in the open position.

FIG. 74 is a right perspective view of another embodiment of an end effector, with the jaws in the closed position.

FIG. 75 is a right perspective view of the end effector of FIG. 74, with the jaws in the open position.

FIG. 76 is a right perspective view of another embodiment of an end effector, with the jaws in the closed position.

FIG. 77 is a right perspective view of the end effector of FIG. 76, with the jaws in the open position.

FIG. 78 is an exploded perspective view of the end effector of FIG. 76.

FIG. 79 is a right perspective view of another embodiment of an end effector, with the jaws in the closed position.

FIG. 80 is a right perspective view of the end effector of FIG. 79, with the jaws in the open position.

FIG. 81 is an exploded perspective view of the end effector of FIG. 79.

### DETAILED DESCRIPTION

Embodiments of a surgical instrument are disclosed for use in a wide variety of roles including, for example, grasping, dissecting, clamping, electrocauterizing, or retracting materials or tissue during surgical procedures performed within a patient's body.

Certain terminology is used herein for convenience only and is not to be taken as a limitation. For example, words such as “upper,” “lower,” “left,” “right,” “horizontal,” “vertical,” “upward,” and “downward” merely describe the configuration shown in the figures. The components may be oriented in any direction and the terminology, therefore, should be understood as encompassing such variations unless specified otherwise.

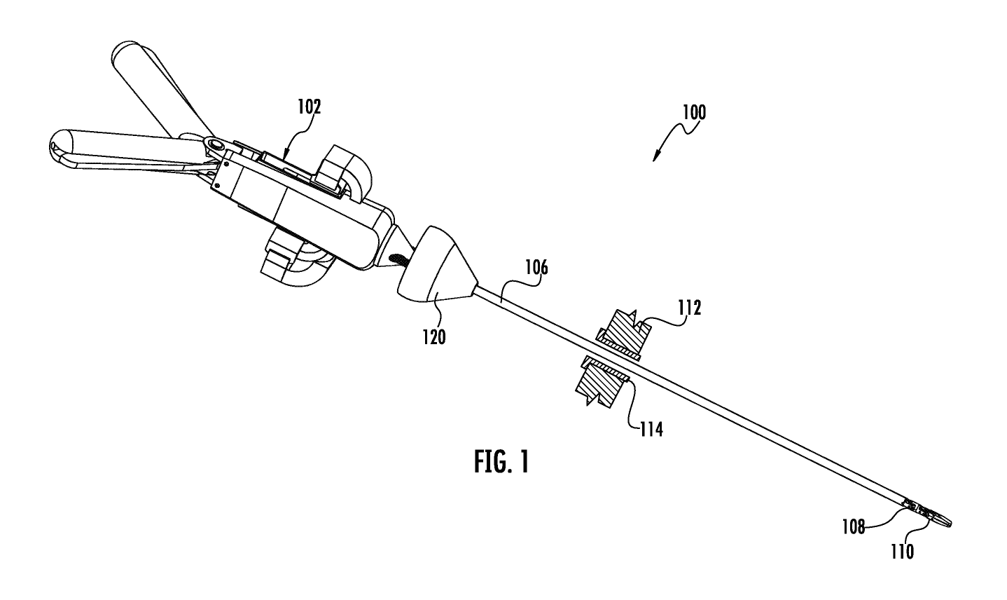

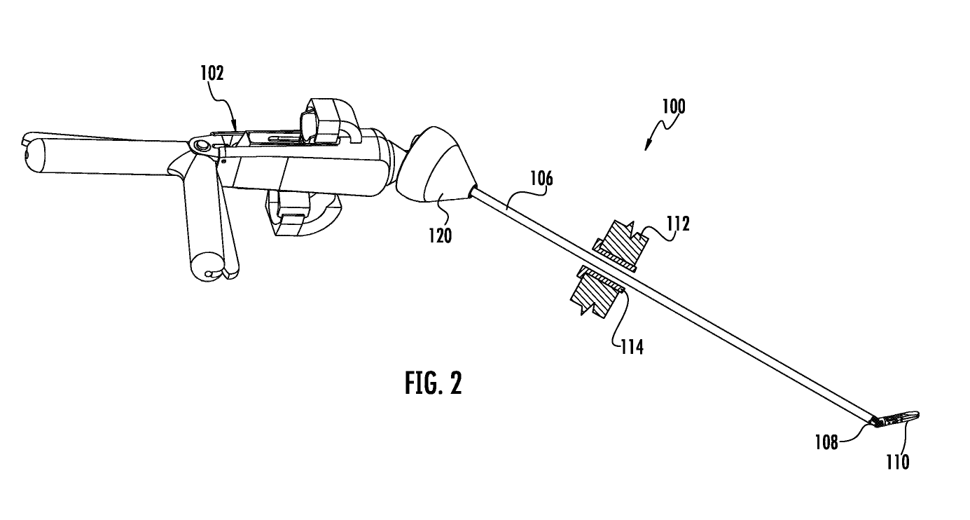

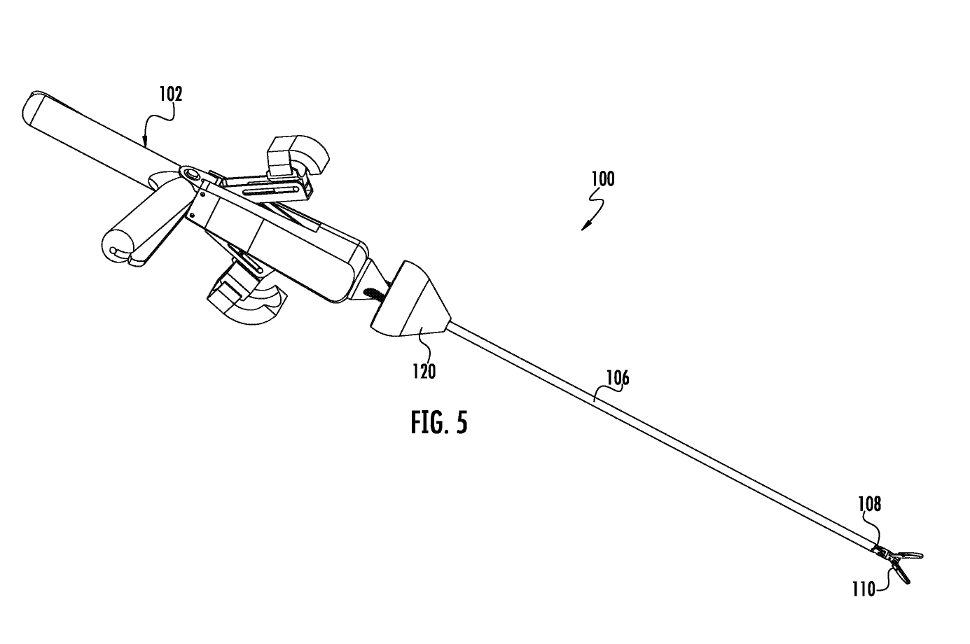

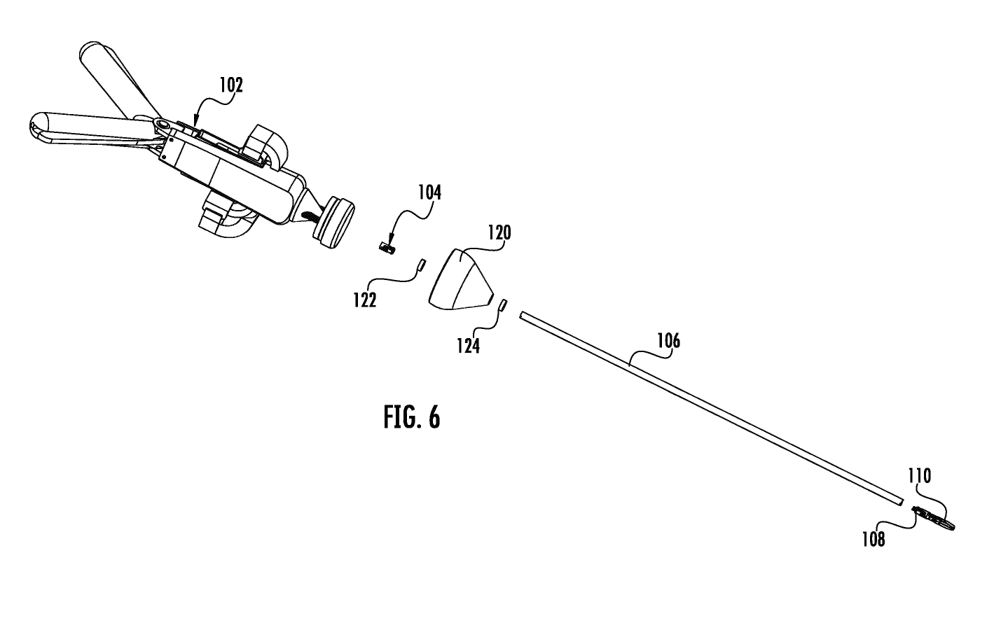

Referring now to the drawings, wherein like reference numerals designate corresponding or similar elements throughout the several views, an embodiment of a surgical tool is shown in FIGS. 1-6 and is generally designated at 100. The surgical tool 100 includes embodiments of five primary components: a manipulator 102, a proximal universal joint 104 (FIG. 6) mounted to the manipulator 102, an elongated, hollow member or tube 106 mounted to the proximal universal joint 104, a distal universal joint 108 mounted to the tube 106, and an end effector 110 mounted to the distal universal joint 108. The manipulator 102 is gripped by a user's hand, with ergonomic features that receive the index finger and the thumb, as described further below. The manipulator 102 and the end effector 110 are operatively connected with cables, as discussed further below, such that when the surgeon moves his finger and thumb to control the manipulator 102, the end effector 110 has corresponding movements. The surgical tool 100 is shown in use in FIGS. 1 and 2, with a portion of the tube 106, the distal universal joint 108, and end effector 110 having passed through a tissue wall 112 via a cannula 114.

FIGS. 1-6 show the surgical instrument embodiment in several different configurations and positions. FIG. 1 shows the instrument in its neutral position, not articulated, with the end effector 108 and manipulator 102 in a closed position. The movement of the proximal universal joint 104, which is attached to the manipulator 102, controls the movement of the distal universal joint 108. The universal joints 104, 108 are operatively connected to each other with cables, as will be discussed further below, and each of the universal joints 104, 108 provide two degrees of freedom, being free to move in any combination of directions deflecting from the longitudinal axis of the tube 106.

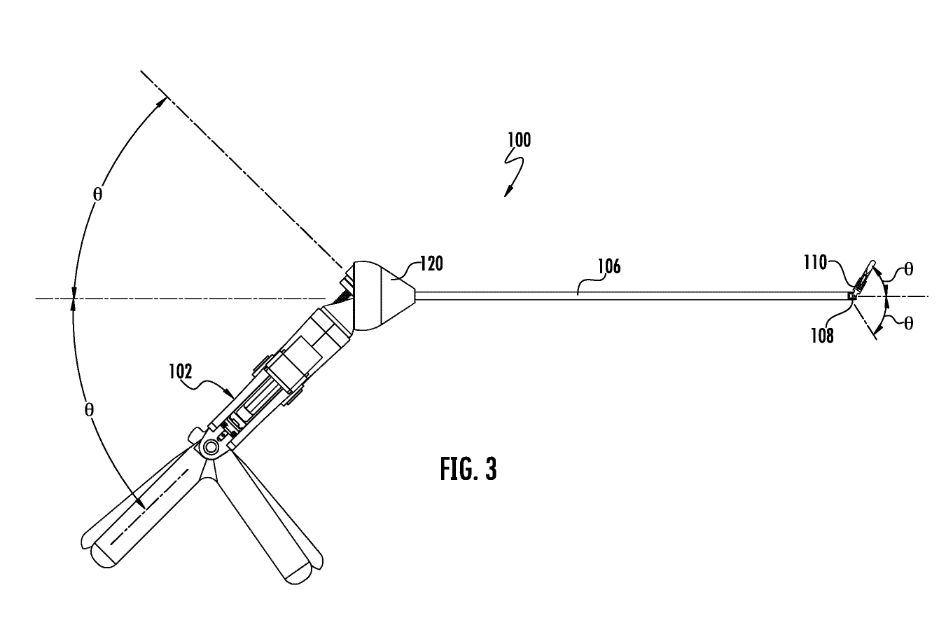

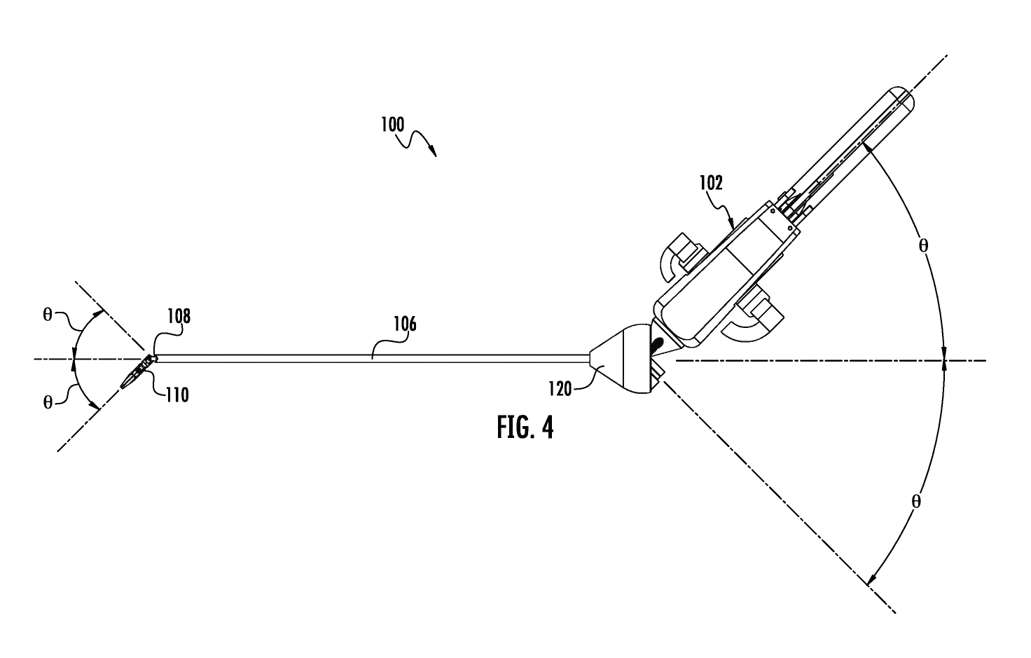

The cabling arrangement enables a surgeon to angle the manipulator 102 with his or her hand relative to the proximal universal joint 104 to cause the distal universal joint 108 to move in a similar manner in the opposite direction, imitating the surgeon's movements and providing directional control of the distal portion of the device. Such corresponding pivoted positions of the manipulator 102 and the end effector 110 relative to the longitudinal axis of the tube 106 are shown in FIGS. 2-4. The maximum angle of deflection θ in every direction from the longitudinal axis of the tube 106, such as side to side in FIG. 3 and between top and bottom in FIG. 4, shows the range of motion at each end of the tool 100, and is determined by the design of the universal joints 104, 108 and the direction of deflection, and may vary from the approximately 45 degrees that is shown. The tube 106 contains the cabling that operatively connects the manipulator 102 to the end effector 110 and the proximal universal joint 104 to the distal universal joint 108.

FIG. 5 shows the instrument 100 with the end effector 110 in an open position. The motion of control assemblies in the manipulator 102 correspond to the motion of elements in the end effector 110 designed to interface with tissue within a patient's body. While the proximal joint 104 effects the orientation of the end effector, the manipulator 102 controls the motion and allows for the manipulation of tissue. FIG. 6 shows the proximal universal joint 104 and a joint guard 120 positioned between two bearings 122, 124.

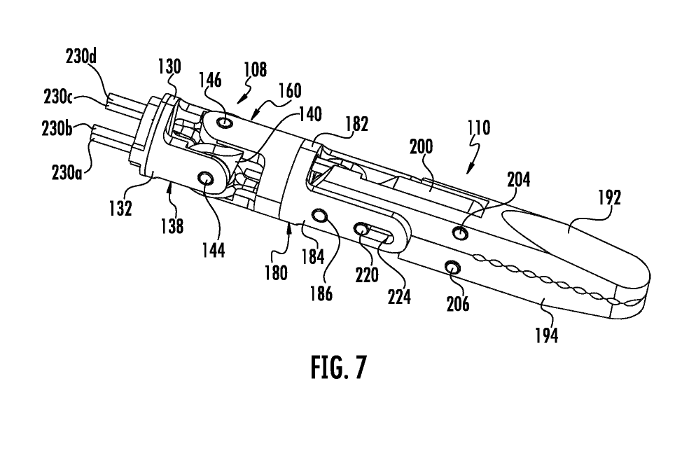

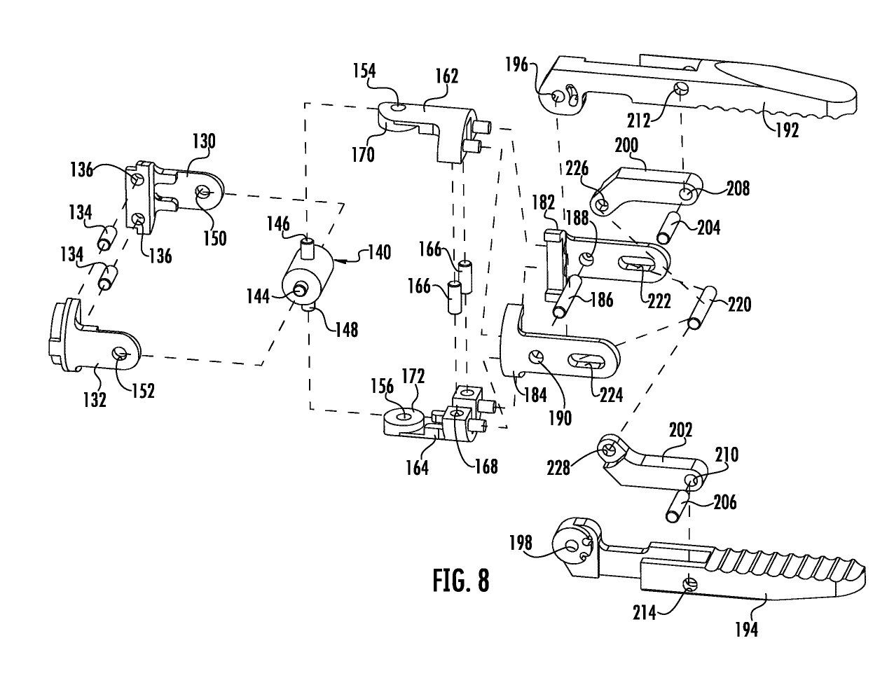

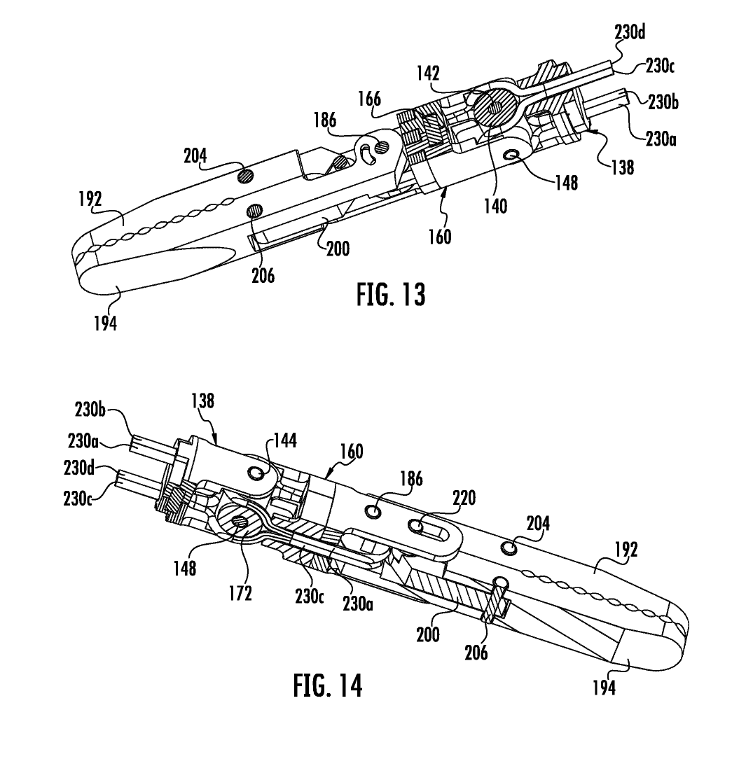

FIGS. 7 and 8 show distal universal joint 108 and end effector 110 embodiments. In the distal universal joint 108 there may be two base elements 130, 132 connected by pins 134 that are disposed in openings 136 to form a proximal yoke 138. The proximal yoke 138 may be constructed in two parts as shown or may be manufactured as a single part. A center block 140, which may be cylindrical as shown or other shape as selected by one of ordinary skill in the art, includes pins 142 (FIG. 13), 144 at each end that are placed in openings 150, 152 in the proximal yoke 138. The pins 142, 144 may be a pair of pins or a single pin passing through the center block 140, and establish a first axis for pivoting of the distal universal joint 108. The center block 140 also includes pins 146, 148 extending from the sides of the center block 140 that are placed in openings 154, 156 in the distal yoke 160. The pins 146, 148 may be a pair of pins or a single pin passing through the center block 140, and establish a second axis for pivoting of the distal universal joint 108. The first axis and the second axis may be intersecting and perpendicular to each other. The pins 142, 144, 146, 148 in the center block 140 may also be pin-like features integrated into the center block, or alternatively may be integrated into the proximal yoke 138 and distal yoke 160, interfacing with holes in the center block 140.

The distal yoke 160 may include two parts 162, 164 connected by pins 166 that extend into openings 168, but may alternatively be manufactured as a single piece. The openings 154, 156 that receive the pins 146, 148 of the center block 140 are disposed centrally and laterally through round features 170, 172 in the arms of the two parts 162, 164 of the distal yoke 160 and allow the distal yoke 160 to pivot about the first and second axes to have two degrees of freedom.

The end effector 110 includes a jaw base 180 that may be two parts 182, 184 as shown, or alternatively one part, and is mounted to the distal yoke 160. Pins 186 of the distal yoke components 162, 164 extend into openings 188 in the two parts 182, 184 of the jaw base 180. The jaw base parts 182, 184 and distal yoke elements 162, 164 may be manufactured in a variety of configurations, for example, as four separate pieces, or as three pieces where two of the original four pieces have been produced as one piece, or as two pieces where two pairs of the original four pieces have each been produced as a single piece, or as two pieces where three of the original four pieces have been produced as a single piece, or as one piece integrating all four original pieces.

A first jaw pin 186 may be mounted to the jaw base 180 at openings 188, 190 and defines a jaw pivot axis. Two jaws 192, 194 are mounted on the first jaw pin 186 at openings 196, 198 near the proximal ends of the jaws 192, 194. Each jaw 192, 194 is connected to a jaw link 200, 202 via a jaw link pin 204, 206 at an opening 208, 210 near the distal end of each jaw link 200, 202 and at an opening 212, 214 at a substantially central location on each jaw 192, 194. A sliding pin 220 is disposed in a slot 222, 224 in each jaw base part 182, 184. The proximal end of each jaw link 200, 202 is mounted to the sliding pin 220 at openings 226, 228 in the jaw links 200, 202. As will be seen, opening and closing the jaws 192, 194 causes the sliding pin 220 to move distally and proximally respectively along the longitudinal axis of the end effector 110. Operation of the jaws 192, 194 and pivoting of the distal yoke 160 and consequently the end effector 110 are brought about by manipulation of the cables 230a, 230b, 230c, 230d.

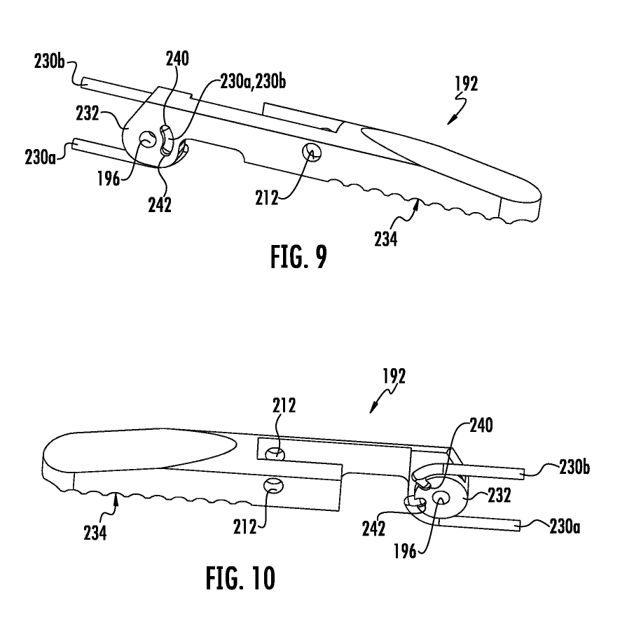

FIGS. 9 and 10 show one jaw 192 with the corresponding control cabling. The jaw 192 has a round feature 232 that acts as a pulley and allows this jaw 192 to rotate on the first jaw pin 186 that passes through the first jaw 192 at a central opening 196 of the round feature 232. The second jaw 194 may be substantially identical to the first jaw 192, as shown. A toothed portion 234 is provided, but alternatively other surface treatments or cutting blades could be provided. There are two holes 240, 242 in the jaw 192 that receive two of the control cables 230a, 230b. These cables 230a, 230b are, as shown, actually a single cable that passes through the two cable holes 240, 242 before continuing in a proximal direction into the end effector 110. The control cables 230a, 230b function separately and can be constructed as one cable as shown or as two cables that terminate and are secured at the first jaw 192. Control cables 230c, 230d that are associated with the second jaw 194 may be configured in a like manner. Effectively, each of the cables 230a, 230b, 230c, 230d may be considered a cable length, whether the cables are continuous or not, so there are four cable lengths, which are referred to as cables herein. In the embodiment shown, friction holds the continuous cables 230a, 230b fixed in the slot on the right side of the first jaw 192. Attachment methods may include, but are not limited to, friction, adhesive, swaged components that apply pressure to cables, or any combination of these methods.

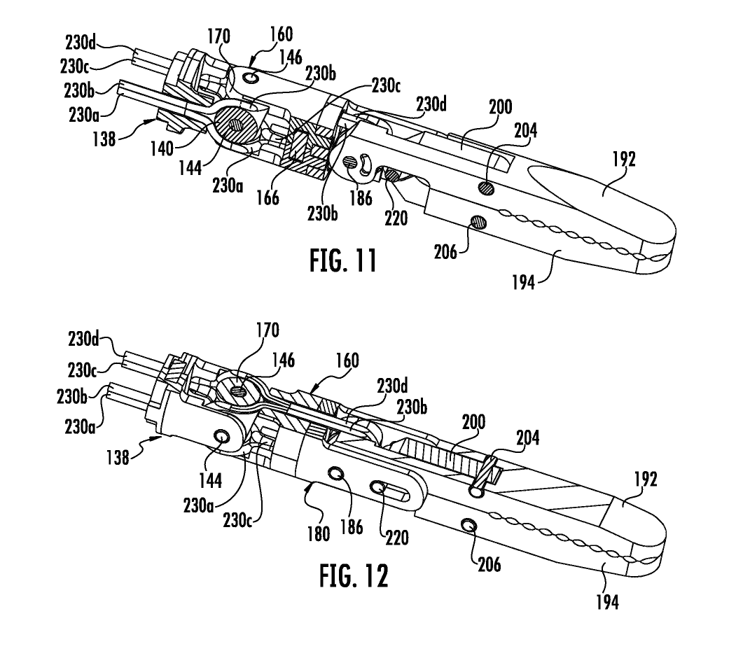

FIGS. 11-14 show how the control cables are routed through the end effector 110. Cables 230a and 230b begin on the first jaw 192, as shown in FIGS. 9 and 10, and cables 230c and 230d begin on the second jaw 194 in a similar manner. Cable 230a passes from the first jaw 192 through the bottom 164 of the distal yoke 160, around a round feature 172 of the distal yoke 160, under the center block 140 and through the proximal yoke 138. Cable 230b passes from the first jaw 192 through the top 162 of the distal yoke 160, around a round feature 170 of the distal yoke 160, over the center block 140 and through the proximal yoke 138. Cable 230c passes from the second jaw 194 through the bottom 164 of the distal yoke 160, around a round feature 172 of the distal yoke 160, under the center block 140 and through the proximal yoke 138. Cable 230d passes from the second jaw 194 through the top 162 of the distal yoke 160, around a round feature 172 of the distal yoke 160, over the center block 140 and through the proximal yoke 138. The previously mentioned round features 170, 172 of the distal yoke 160 may be manufactured in various configurations including, but not limited to, being idling pulleys separate from the distal yoke 160 or features of the center block 140.

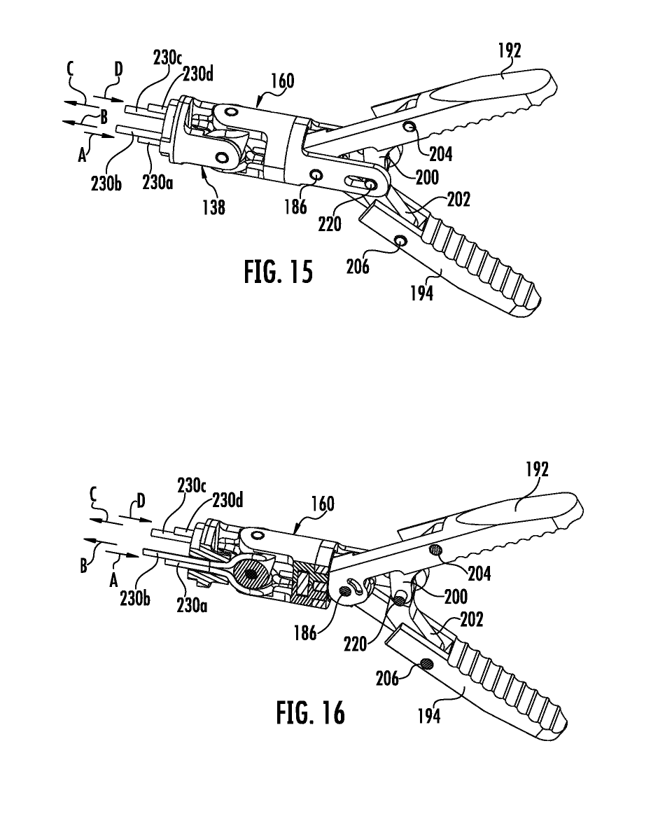

FIGS. 15 and 16 show the distal universal joint 108 and end effector 110 with jaws 192, 194 in an open position, along with the corresponding cabling. A movement in which two cables are retracted in a proximal direction and two cables are relaxed in a distal direction will be denoted in a format hereinafter as WX/YZ linear motion of cables, where W and X represent the proximally moving (retracted) cables and Y and Z represent the distally moving (extended) cables. Linear motion of cable 230a is denoted by A; linear motion of cable 230b is denoted by B; linear motion of cable 230c is denoted by C; and linear motion of cable 230d is denoted by D. A BC/AD motion produces the effect of opening the jaws. Since diagonally opposed cables B and C are retracted, there is no effect on either of the axes of the yokes 138, 160 of the distal universal joint 108. As the BC/AD motion opens the jaws 192, 194, the sliding pin 220 moves distally via the jaw links 200, 202 and associated pins 204, 206.

FIG. 17 further depicts the means by which the proximal universal joint 104 controls the distal universal joint 108 and end effector 110. Four cables 230a, 230b, 230c, 230d connect the two joints 104, 108, are fixed at both ends, and control the motion of the universal joints 104, 108 about their two primary axes, as established, for example, by the pins 142, 144, 146, 148 (FIGS. 11-14) in the distal universal joint 108. As shown, the proximal universal joint 104 may be configured like the distal universal joint, with the yokes reversed, i.e., the distal yoke 250 of the proximal universal joint 104 may be similar to the proximal yoke 138 of the distal universal joint 108, and the proximal yoke 252 of the proximal universal joint 104 may be similar to the distal yoke 160 of the distal universal joint 108. The center block 254 of the proximal universal joint 104 may be, as shown, similar to the center block 140 of the distal universal joint 108. The configuration of the cabling in the proximal universal joint 104, also as shown, may be a mirror image of that in the distal universal joint 108.

With respect to the proximal universal joint 104, the ends of the cables 230a, 230b, 230c, 230d are fixed via a set of tensioning assemblies in the manipulator 102, discussed further below. This allows the relative positioning of the proximal and distal universal joints 104, 108 to be calibrated during manufacturing.

Exemplary operational scenarios are as follows. As previously noted, in FIG. 17, upper case letters again denote motion of a cable, and retraction of two cables derived from a pivoting motion of the proximal yoke 252 of the proximal universal joint 104 causes a pivoting motion of the distal yoke 160 of the distal universal joint 108. Retraction of diagonally opposed cables results in a motion of the jaws 192, 194 of the end effector 110. When the proximal yoke 252 of the proximal universal joint 104 pivots 260 about the proximal center block 254 in a counterclockwise direction (designated CD), then cables 230c and 230d are displaced downward and cables 230a and 230b are displaced upward. This produces a similar pivot 262 in the counterclockwise direction CD of the distal yoke 160 of the distal universal joint 108 about the distal center block 140. With respect to rotation in a perpendicular plane to motion 260, when the proximal yoke 252 of the proximal universal joint 104 pivots 264 about the proximal center block 254 in a counterclockwise direction (designated BD), cables 230b and 230d are displaced downward and cables 230a and 230c are displaced upward. This produces a similar pivot 266 in the counterclockwise clockwise direction BD of the distal yoke 160 of the distal universal joint 108 about the distal center block 140 relative to the proximal yoke 138.

Motion 264 in clockwise direction AC in the proximal universal joint 104 likewise causes motion 266 in clockwise direction AC in the distal universal joint 108, and motion 260 in clockwise direction AB in the proximal universal joint 104 causes motion 262 in clockwise direction AB in the distal universal joint 108. The various motions may be combined. The mounting of the proximal yoke 252 of the proximal universal joint 104 to the distal end of the manipulator 102 results in the movement of the manipulator 102 causing the movement of that yoke 252. In the embodiment shown, all motions of the proximal yoke 252 of the proximal universal joint 104 actuate cables 230a, 230b, 230c, 230d to produce similar motion in the opposite direction in the distal yoke 160 of the distal universal joint 108. In addition, as described with respect to FIGS. 15 and 16, cable motions BC/AD open the jaws 192, 194, and this result is shown in FIG. 17 at pivot motion 268 of one jaw 194.

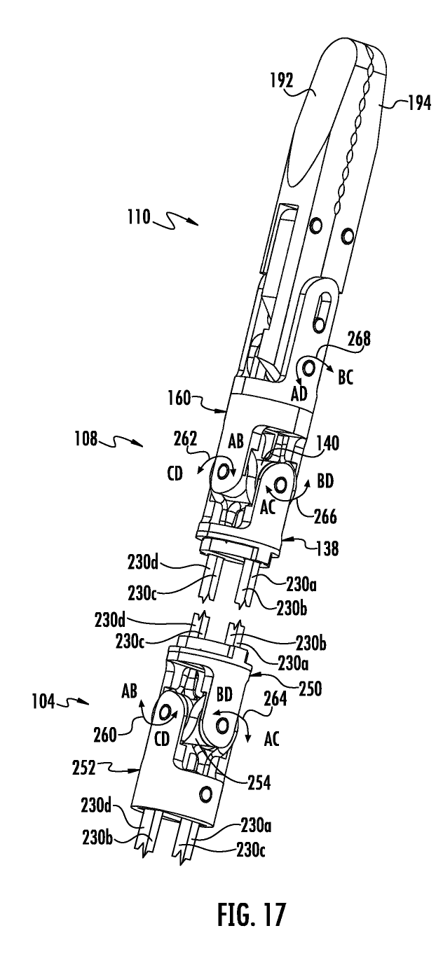

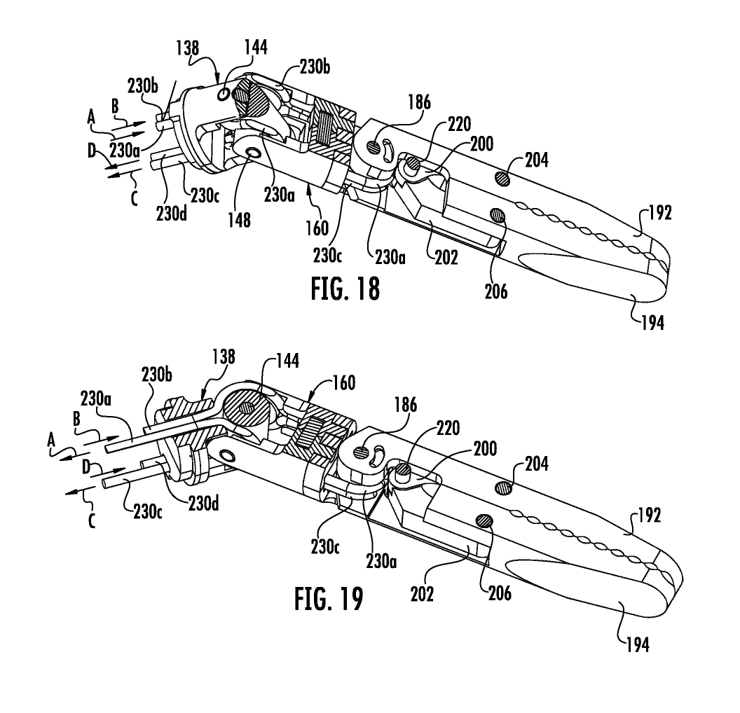

FIG. 18 shows the distal universal joint 108 articulated along its second axis as defined by the distal yoke pins 146 (not visible), 148. This is accomplished by a CD/AB motion. Since there is no relative motion A, B of cables 230a and 230b to each other, the first jaw 192 is not affected. Similarly, there is not relative motion C, D of cables 230c and 230d to each other, so the second jaw 194 is unaffected. Thus, this motion produces articulation along the second axis of the distal universal joint 108 by rotating the distal yoke 160 and end effector 110 about the applicable distal yoke pins 146, 148.

FIG. 19 shows the distal universal joint 108 articulated along its first axis as defined by the proximal yoke pins 142 (not visible), 144. This is accomplished by an AC/BD cable motion. The relative motion C, D of cables 230c and 230d acts to produce this articulation, but also attempts to produce an opening motion of the second jaw 194. The relative motion A, B of cables 230a and 230b acts to produce articulation about the first axis, but also attempts to produce a closing motion of the first jaw 192. The opening motion of the second jaw 194 would cause a distal motion of the sliding pin 220, while the closing motion of the first jaw 192 would cause a proximal motion of the sliding pin 194. Thus, the linkage system including the jaws 192, 194, the sliding pin 220, and jaw links and pins 200, 202, 204, 206 braces against any opening or closing effect that the AC/BD motion may have produced and there is no effect on the jaws 192, 194.

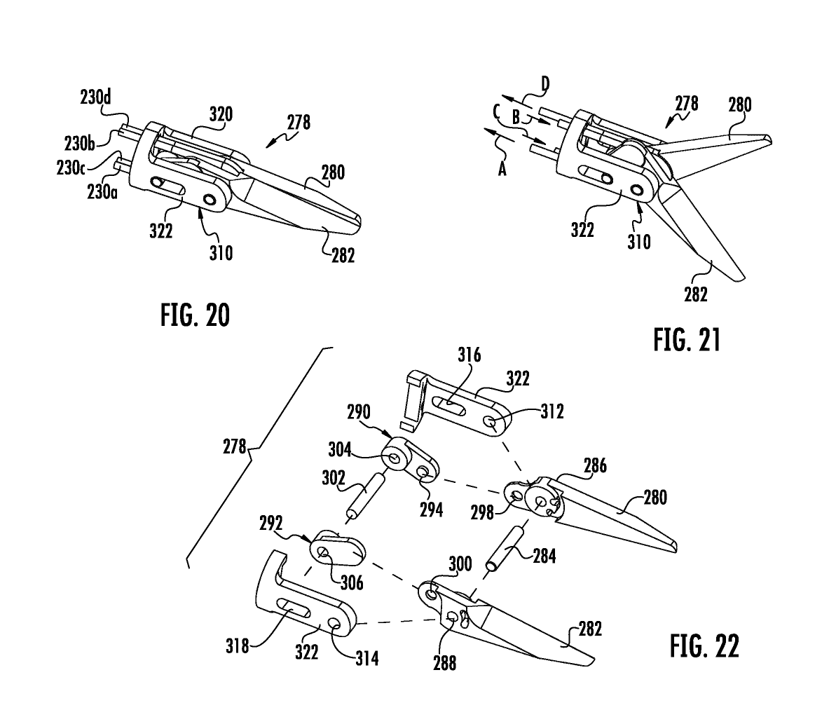

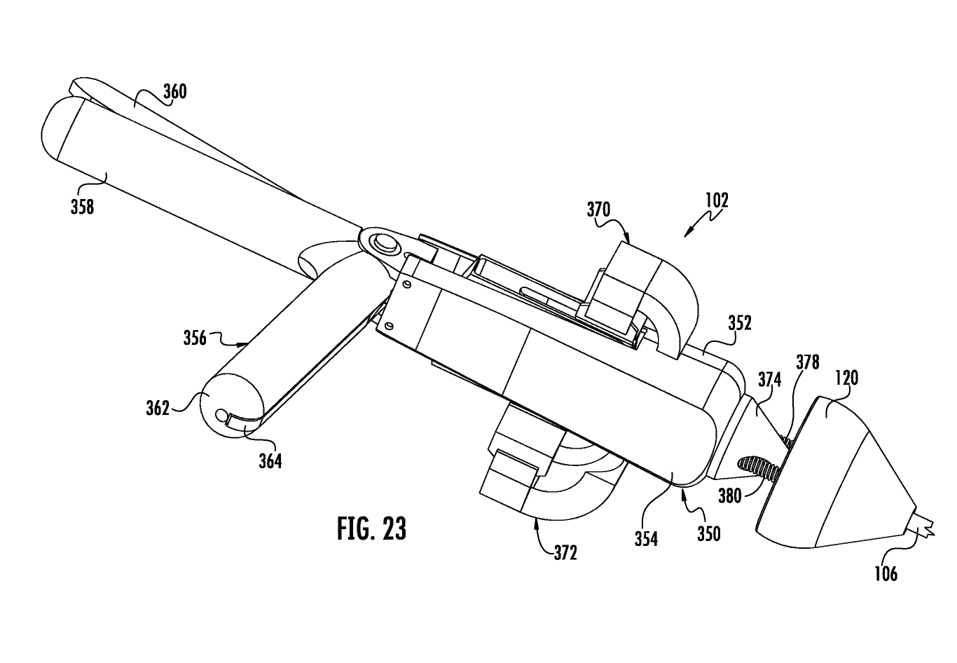

Jaws 192, 194 may be replaced with scissor blades or other implements in certain embodiments. The jaws may be of any of a variety of configurations. They may be tailored to a specific task, such as suture grasping, tissue grasping, tissue dissection, tissue cutting, or electrocautery. In general, the end effector 110 may be replaced by any other embodiment in which two jaws are controlled by pairs of cables 230a, 230b and 230c, 230d in a manner such that the jaws are permitted to rotate in opposite directions but prevented from moving in the same direction. One such embodiment of an end effector 278 is shown in FIGS. 20-22, in which an end effector in which the jaws 192, 194 are replaced with scissor blades 280, 282 to produce a scissors apparatus. In this embodiment, cables connect to the blades 280, 282 which are mounted on a first jaw pin 284 at openings 286, 288 in the blades 280, 282. The blades connect to constraining links 286, 288 via pin-like features 294 that may be manufactured as part of the constraining links 286, 288 as shown or may be separate pins that are inserted into the constraining links 286, 288. The pin-like features 294 are inserted into openings 298, 300 at the proximal ends of the blades 280, 282. The constraining links 286, 288 also connect to a sliding pin 302 at openings 304, 306. The blades 280, 282 and constraining links 286, 288 are mounted to the jaw base 310 with the first jaw pin 284 and the sliding pin 302 that extend through openings 312, 314 and slots 316, 318 in the jaw base parts 320, 322, respectively. When the first blade 280 rotates counterclockwise, the first constraining link 290 rotates clockwise and moves in a distal direction, forcing the sliding pin 302 and second constraining link 306 to move distally, which rotates the second blade 282 clockwise. Thus, the blades 280, 282 are constrained to move in opposite directions. In contrast to the previously described embodiments, this assembly is actuated to open by an AD/BC motion rather than a BC/AD motion. However, this is difference would not affect an embodiment of the distal universal joint 108 attached to this jaw 278 design, though it would slightly alter the cabling configuration in the manipulator 102, as will be discussed further.

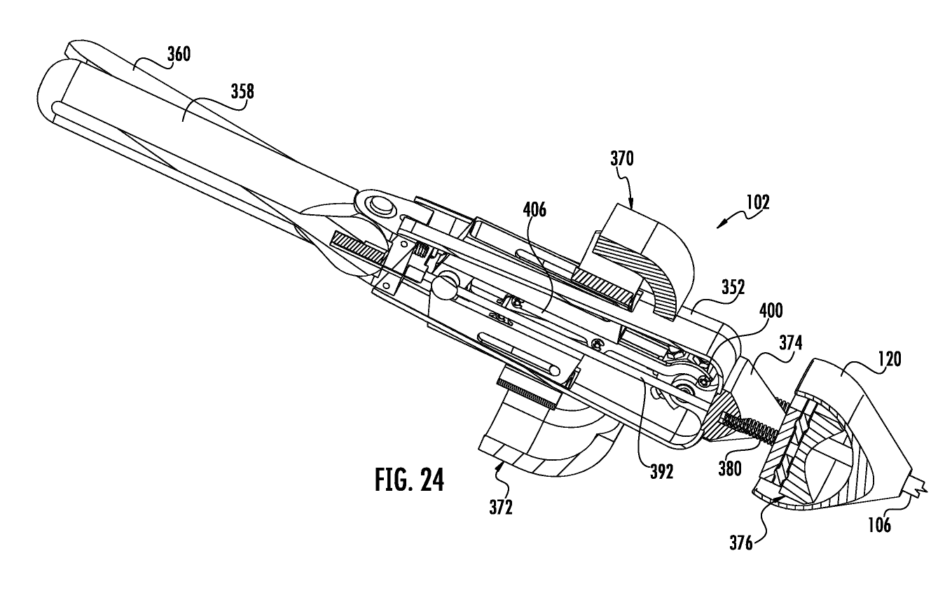

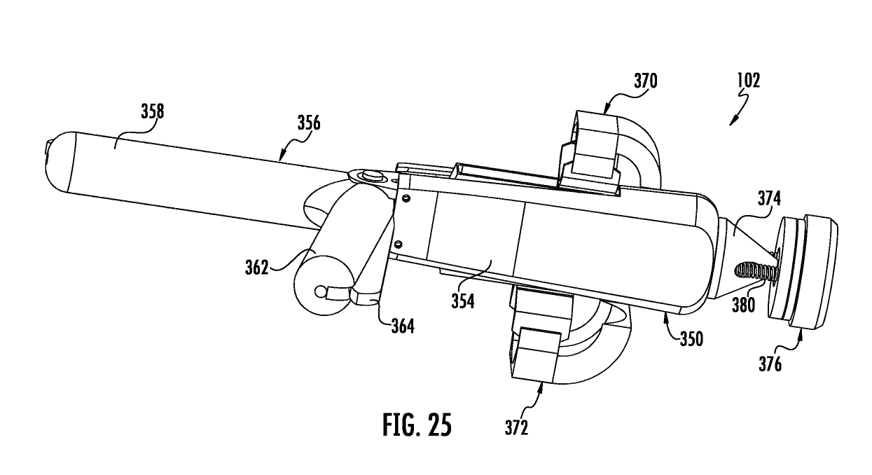

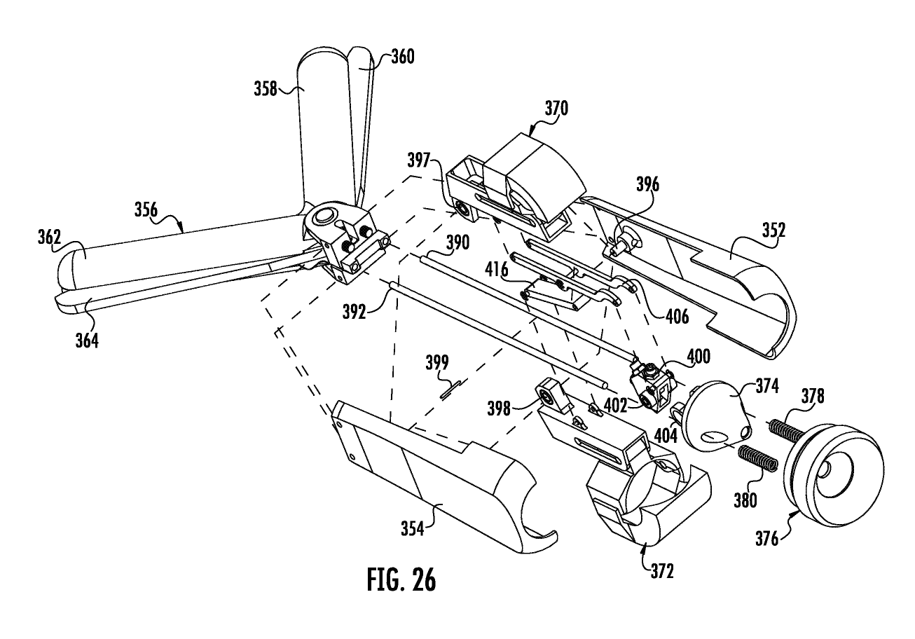

FIGS. 23 and 24 show the proximal end of the instrument 100 including the manipulator 102, the joint guard 120, and the elongated tube 106. The manipulator 102 includes, as also shown in FIGS. 25 and 26, a housing 350 in two parts 352, 354, a handlebar assembly 356 including a left handle 358 with a left trigger 360, a right handle 362 with a right trigger 364, an index assembly 370, a thumb assembly 372, and a joint adapter 374 mounted to the housing 350. The manipulator 102 is shown configured in a right-handed orientation, i.e., for a user's right index finger to be inserted in the index assembly 370, the user's right thumb to be inserted in the thumb assembly 372, and the user's remaining fingers on the right hand to grasp the right handle 362, but the configuration may be altered to a left-handed configuration by pivoting of the handlebar assembly 356. In FIGS. 23 and 24, the right trigger 364 is operational with the user's remaining fingers, is in a retracted position, being substantially contained within the handlebar 362, and is responsible for controlling the movement of the brake assembly 376 (FIGS. 25 and 26). In FIG. 25 the trigger 364 is not actuated. The brake assembly 376 is biased by two springs 378, 380 to press against the joint guard 120, which locks the articulation of the proximal joint 104. When the user wants to articulate the instrument 100, the trigger 364 is depressed, which retracts the brake assembly 376 via brake rods 390, 392. The brake rods 390, 392 interface with the handlebar assembly 356 via an interface bar 394, and the trigger 364 controls the interface bar 394 in a manner further described below. The joint adapter 374 holds the proximal joint 104 and also limits the maximum angle that the manipulator 102 can be articulated from the longitudinal axis of the tube section 106.

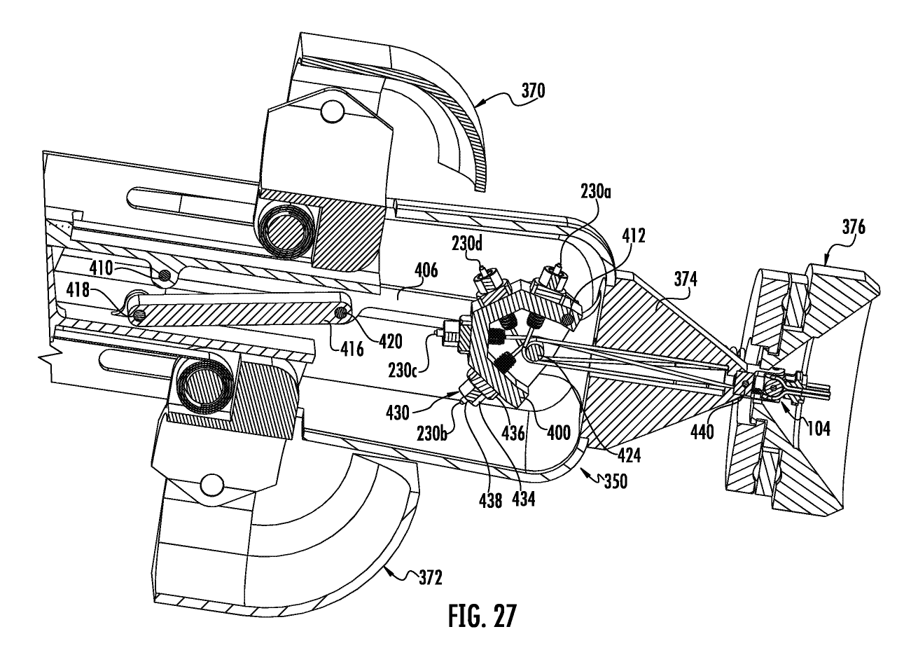

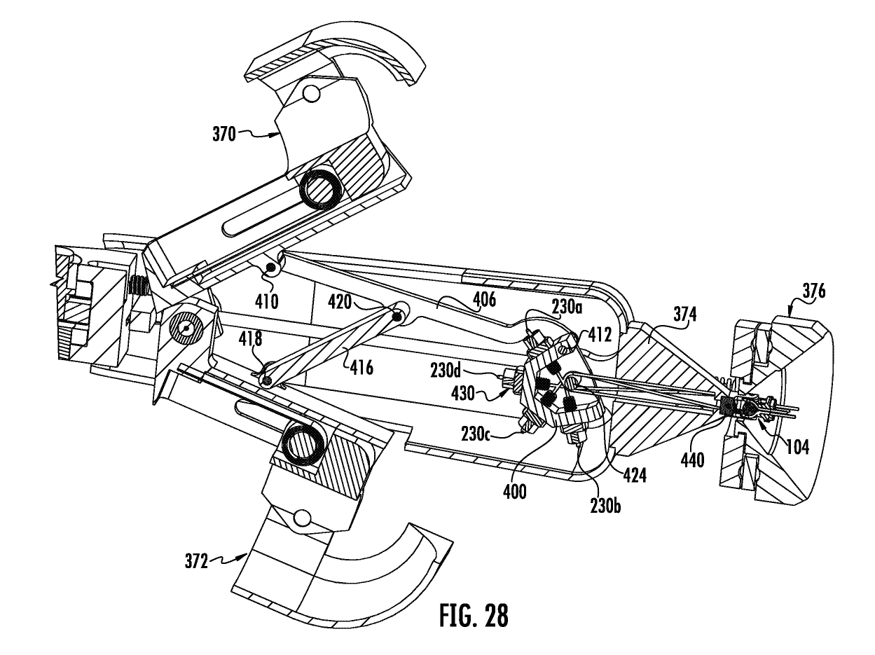

FIGS. 26-28 show internal components of the manipulator 102. A anchor 400 is provided that is pivotally mounted to the joint adapter 374 at two ball bearings 402 placed in openings 404 of the joint adapter 374. The anchor 400 is shaped generally as a “U” in longitudinal section (FIGS. 27 and 28), with an opening at the distal end to receive cables 230a, 230b, 230c, 230d, and webs to enclose the sides.

A round feature on the inside of each housing part 352, 354, only one of which round features 396 is visible, is inserted into bearings at openings 397, 398 in the thumb assembly 372 and index assembly 370 along with a pin 399 to secure the assemblies 370, 372 to the housing 350. The index assembly 370 connects to the anchor 400 via the index link 406 and two pins 410, 412 at the ends of the index link 406. The index link 406 may include parallel elongated members with a substantially central transverse member. The thumb assembly 372 connects to the index link 406 via the thumb link 416 and two pins 418, 420 at the ends of the thumb link 416. The thumb link 416 may include an elongated member that is disposed at its connection to the index assembly 106 between the elongated parallel members of the index link 406. In this manner, the thumb assembly 372 and index assembly 370 are constrained to move in opposite directions while actuating the anchor 400. The anchor 400 pivots about a shaft 424 between its bosses 402 and actuates the control cables 230a, 230b, 230c, 230d.

As previously noted, cables 230a, 230b, 230c, 230d are routed through the proximal universal joint 104 in the same manner, but in a mirror orientation, as through the proximal yoke 138 and distal yokes 160 and center block 140 of the distal universal joint 108. Each cable terminates in one of four tensioning assemblies 430. The tensioning assemblies 430 may achieve anchoring by means of vented screws 434, nuts 436, and swaged tubing 438, as identified at the ends of cable 230b in FIG. 27. The swaged tubing 438 is compressed onto the control cables to act as mechanical retention against the head of the vented screws 434. Tension is applied to the control cables by rotating the nut 436 while keeping the corresponding vented screw 434 in a constant rotational position. This produces linear translation of the vented screw 434 and a corresponding change in tension in its control cable.

Cables 230b and 230d exit from the top of the proximal end of the proximal joint 104 after passing over a guide pulley 440, while cables 230a and 230c exit from the bottom of the proximal joint 104 after passing under the same guide pulley 440. Cables 230c and 230d cross before entering the anchor 400. The cables 230a, 230b, 230c, 230d are arranged within the anchor 400 such that a counterclockwise rotation of the anchor 400 produces a BC/AD motion, as shown in FIG. 28, which opens the previously described embodiments of the end effector 110. For embodiments where a AD/BC motion is required to open the jaws of the end effector, cables 230a and 230b would cross before entering the anchor 400 and cables 230c and 230d would remain straight.

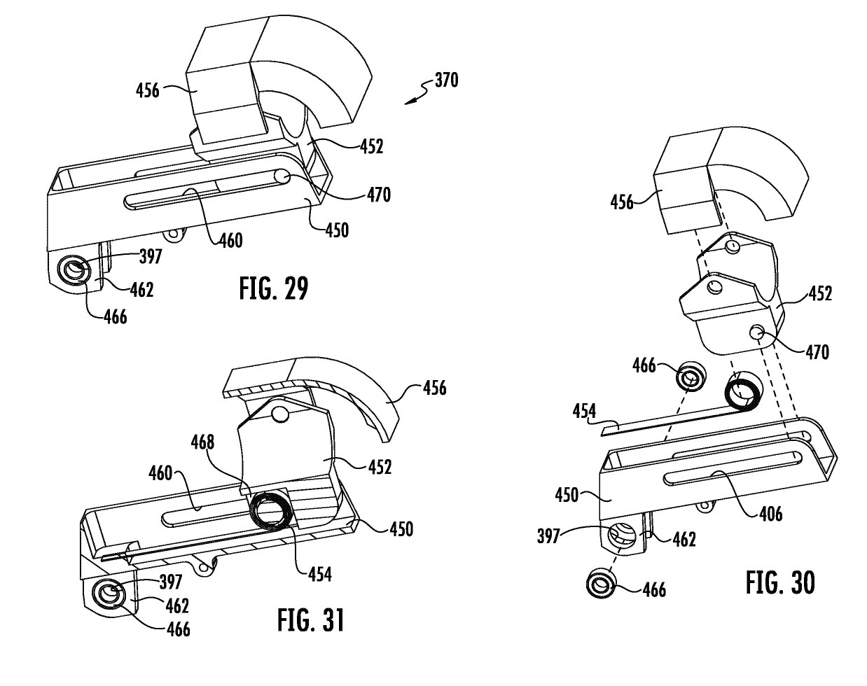

FIGS. 29-31 show the index assembly 370. The thumb assembly 372 is similarly designed with parts suited for accommodating a thumb rather than an index finger. The index assembly 370 includes an index channel 450, a sliding element 452, a spring 454, and a grip 456. The index channel 454 includes a bottom, and end wall, and two parallel sides extending from the bottom with opposed longitudinal slots 460. Parallel tabs 462 extend from the bottom of the index channel 452 and define openings 397 in which bearings 466 are disposed. The index assembly 370 pivots about this pair of bearings 466 that are mounted to round features 396 in the housing 350 of the manipulator 102. The spring 454, which may be a coiled constant force spring with the coil received in a recessed area 468 in the sliding element 452, biases the sliding element 452 along the index channel 450, with protrusions 470 extending laterally from the sliding element 452 sliding in the slots 460. The sliding element 452 can translate along the longitudinal axis of the index channel 450 to accommodate differently sized fingers.

The user inserts an index finger into the opening formed by the sliding element 452 and grip 456. Force is applied to the sliding element 452 by the spring 454, causing the grip 456 to press against the tip of the user's index finger. This exerts a counterclockwise torque on the grip 456, which forces the top of the grip 456 to press against the top of the user's index finger, securing the finger. Thus, the index assembly 370 automatically compensates for variations in finger size and allows the user to engage the instrument without the use of Velcro straps or other means of securing the instrument to their hand. This mechanism allows for one-handed operation of the instrument throughout its use.

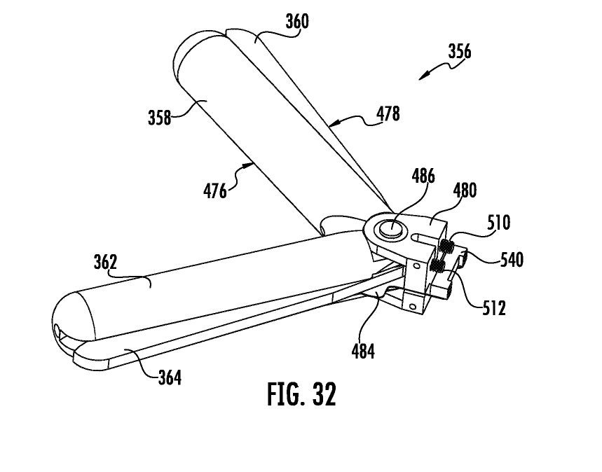

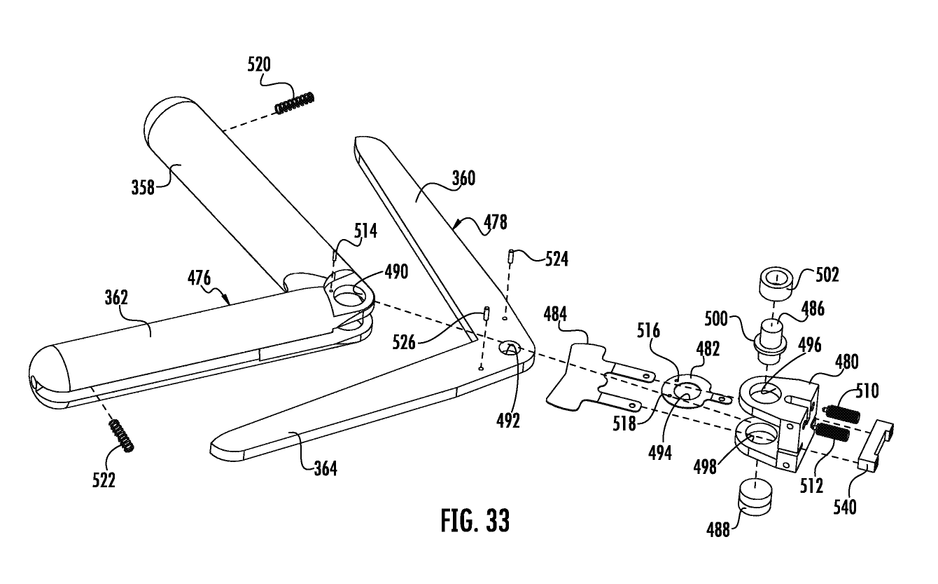

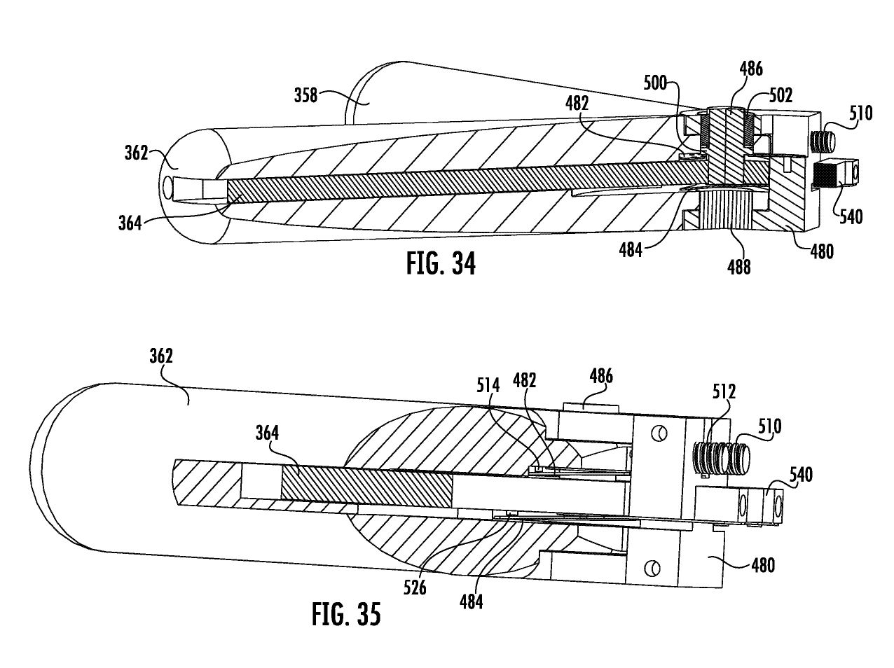

FIGS. 32-35 show the handlebar assembly 356 in its neutral configuration. In addition to the handles 358, 362, which forms the handlebar 476, and the triggers 360, 364, which as shown may be formed from one substantially V-shaped member trigger bar 478 to be received in the handlebar 476, the handlebar assembly 356 includes a base 480, a handlebar lock 482, a trigger lock 484, and a release button 486.

The handlebar 476, trigger bar 478, and handlebar lock 482 are mounted to the base 480 with the release button 486 and handlebar rod 488 that pass through respective openings 490, 492, 494 and through the openings 496, 498 in the base 480. The release button 486 may be cylindrical and includes a substantially central flange 500. A bearing sleeve 502 is disposed in the opening 496 around the upper portion of the release button 486.

Two spring plungers 510, 512 extend through openings in the distal face of the base 480 and apply pressure to the handlebar 476 to bias it towards the neutral position. In this position, the handlebar lock pin 514 rests on the top of the handlebar lock 482. The handlebar lock 482 in this embodiment may be a piece of spring steel that is slightly bent when the handlebar 476 is in the neutral configuration. The handlebar lock 482 can lock the handlebar 476 in either a right-handed or left-handed configuration. When the handlebar 476 is moved into one of these configurations, the handlebar lock pin 514 enters one of the pinholes 516, 518 of the handlebar lock 482. Two springs 520, 522 bias the trigger bar 478 into its neutral position where it is centered with respect to the handlebar 476. The trigger lock pins 524, 526 rest on top of the trigger lock 484 when the handlebar 476 is in its neutral configuration. The trigger lock 484 in this embodiment may also be a piece of spring steel that is slightly bent when the handlebar 476 is in its neutral configuration. The trigger lock 484 may be shaped with a body with two spaced, parallel, elongated tabs extending distally therefrom to pass through two slots in the base 480 and connect to the interface bar 540. When the handlebar 476 is moved into either a right-handed or left-handed configuration, one of the trigger lock pins 524, 526 moves off the front edge of the trigger lock 484 while the other is left behind the trigger lock 484. This allows the trigger lock 484 to unbend, and whichever pin 524, 526 moved off the front edge of the trigger lock 484 can be translated in a proximal direction by depressing the trigger bar 478, which in turn will translate the trigger lock 484 in a proximal direction.

The bearing sleeve 502 and handlebar rod 488 provide surfaces around which the handlebar 476 can pivot, and the end of the release button 486 provides a surface around which the trigger bar 478 can pivot. The flange 500 of the release button 486 rests on top of the inner edge of the handlebar lock 482 and the bottom of the release button 486 rests on top of the trigger lock 484 such that both locks may be deflected downward by a downward translation of the release button 486.

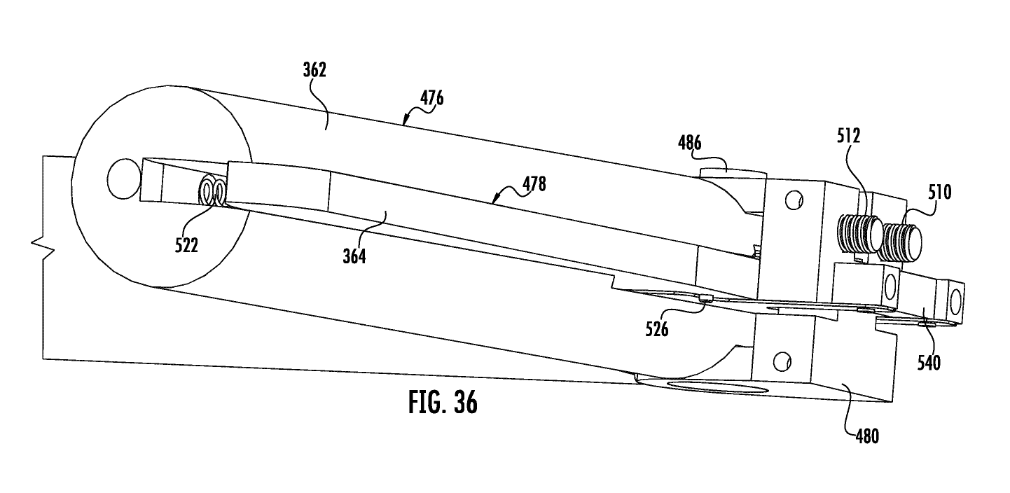

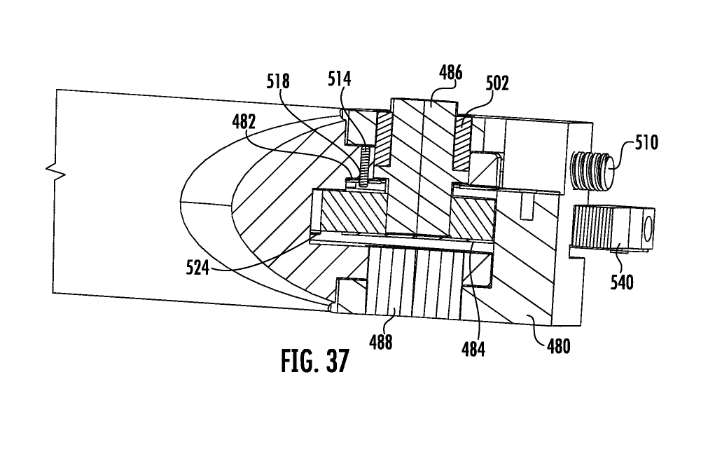

FIGS. 36 and 37 show the handlebar assembly 356 in its right-handed configuration with the trigger bar 478 in its non-actuated position relative to the handlebar 476. In this position, the trigger lock pins 524, 526 no longer rest on top of the trigger lock 484. As such, the trigger lock 484 has become unbent. The right trigger lock pin 526 is in front of one edge of the trigger lock 484 and the left trigger lock pin 524 is behind the trigger lock 484. If the handlebar 476 were in its left-handed configuration, the left trigger lock pin 524 would be in front of a corresponding edge of the trigger lock 484 and the right trigger lock pin 526 would be behind the trigger lock 484. The handlebar lock pin 514 no longer rests on top of the handlebar lock 482 which is now unbent. The handlebar lock pin 514 now rests within the right pinhole 518 of the handlebar lock 482. This locks the handlebar 476 in its right-handed configuration. If the handlebar 476 were in its left-handed configuration, the handlebar lock pin 514 would be in the left pinhole 516 of the handlebar lock 482. Translating the release button 486 downward from its position in either configuration deflects both the handlebar lock 482 and trigger lock 484 downward, releasing them to return to the neutral configuration.

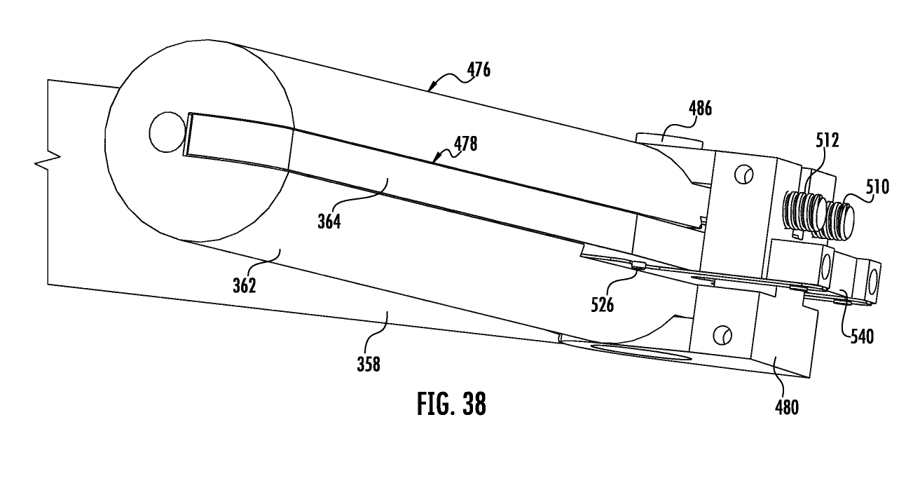

FIG. 38 shows the handlebar assembly 376 in its right-handed configuration with the trigger 364 actuated. This moves the right trigger lock pin 526 such that the trigger lock 484 is translated in a proximal direction, which in turn effects a proximal motion of the brake rods 390, 392 (FIG. 26) and the brake assembly 376 via the interface bar 540. If the handlebar assembly 356 were in its left-handed configuration, then the left trigger lock pin 524 would effect a similar translation.

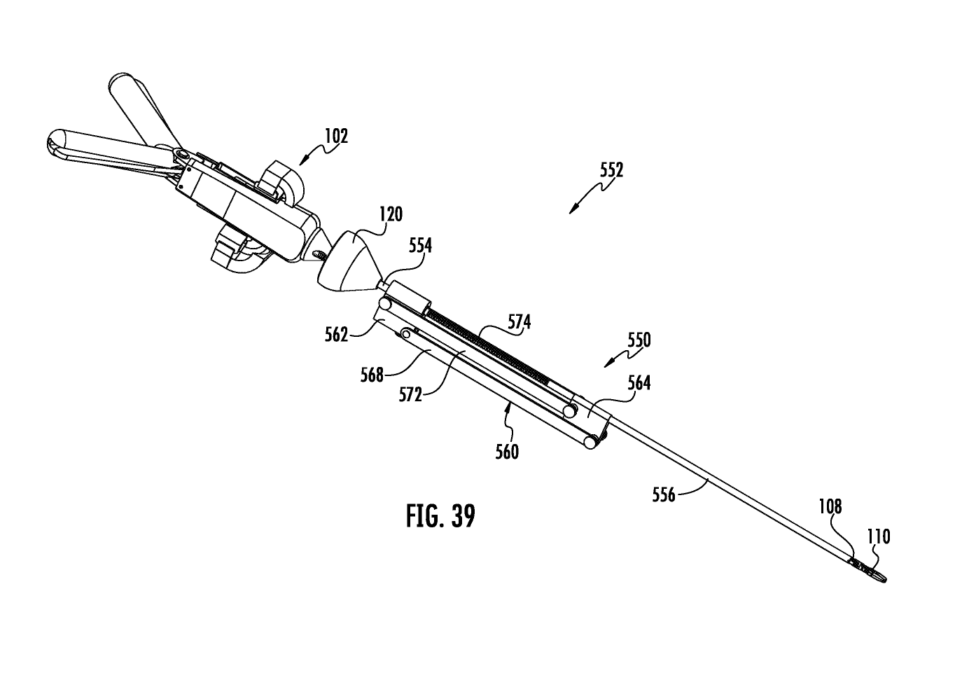

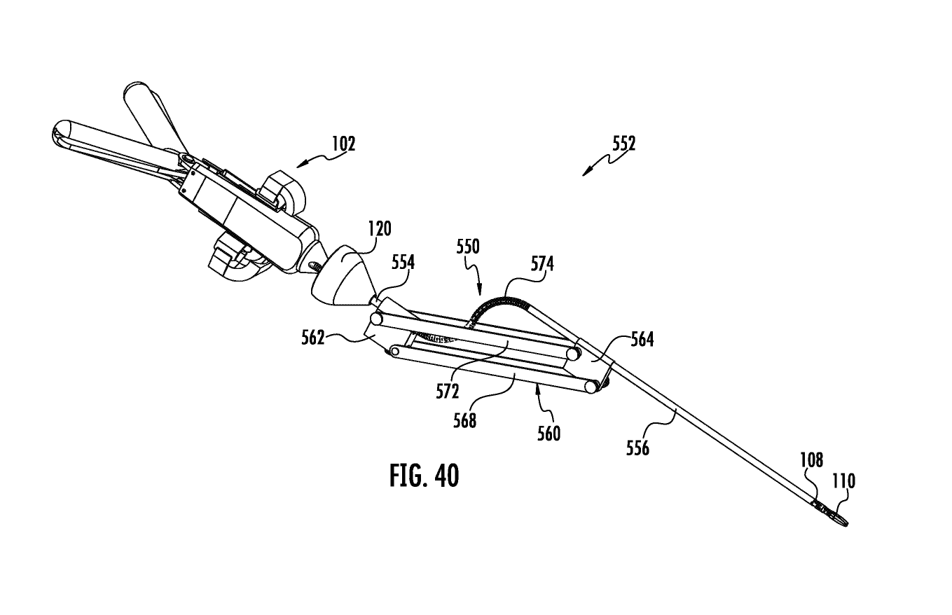

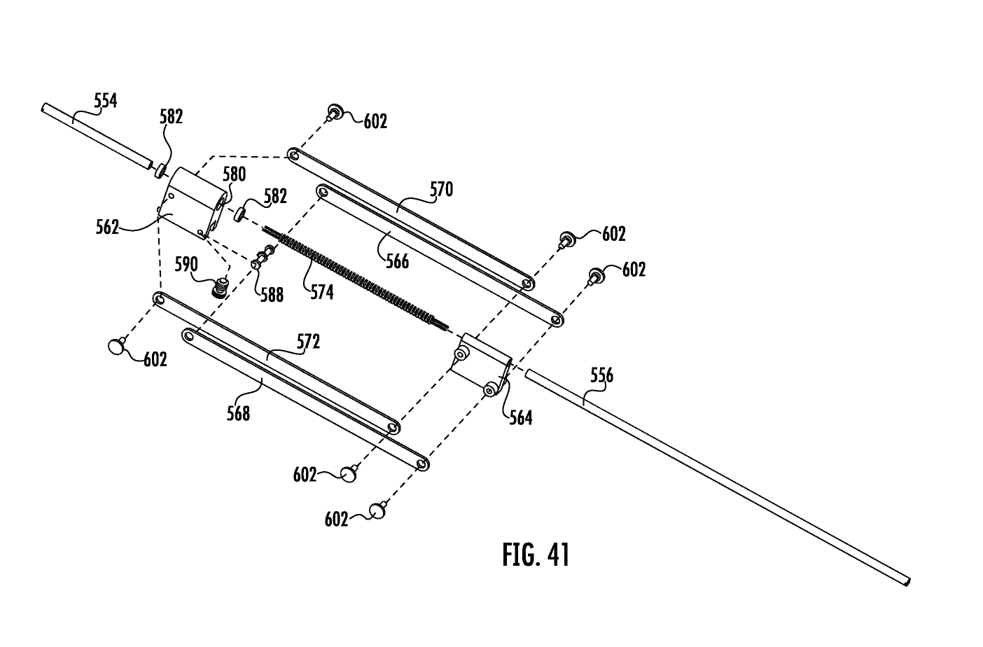

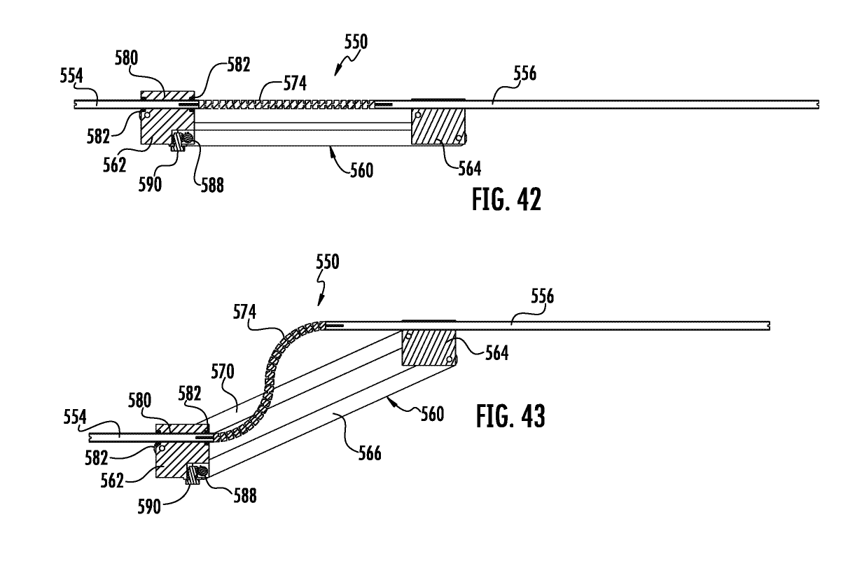

FIGS. 39-43 show an alternate embodiment of the tube section 550 of the instrument 552. This embodiment contains a proximal tube 554 and a distal tube 556 connected by a tube offset assembly 560. This assembly 560 allows the manipulator 102 and proximal joint 104 to be moved laterally such that the longitudinal axis of the proximal tube 554 is parallel to the distal tube 556, and this translation does not interfere with the manipulator's ability to control the distal joint 108 and end effector 110.

The tube offset assembly 560 includes a primary offset base 562, a secondary offset base 564, two actuating links 566, 568, two idling links 570, 572, and a flexible offset element 574. The proximal tube 554 extends through an opening 580 in the primary offset base 562, and is secured in place by a pair of bearings 582. The distal tube 556 extends through an opening 584 in the secondary offset base 564 such that the tube 556 can both rotate and translate within this base 564.

The primary offset base 562 contains an offset drive shaft 588 and an offset driver 590. The two actuating links 566, 568 are connected to the offset drive shaft 588. The two idling links 570, 572 are connected to the primary offset base 562 by bushings 602. All four links 566, 568, 570, 572 are connected to the secondary offset base 564 via bushings 602. When the offset driver 590 is rotated, threads on the offset driver 590 engage teeth on the offset drive shaft 588, causing a corresponding rotation of the offset drive shaft 588 which in turn rotates the actuating links 594, 596, moving the secondary offset base 564 into an offset configuration. This system drives the rotation of the offset drive shaft 588 in such a manner that it may be adjusted and locked at a certain angular position. In this embodiment, the locking effect is achieved via a non-backdrivable gear system.

The proximal tube 554 and distal tube 556 are connected by the flexible offset element 574, through which all four control cables pass. The distal tube 556 passes through the secondary offset base 564. The normal rotations that the manipulator 102 would perform within a cannula during surgery are transmitted from the proximal tube 554 to the distal tube 556 via the flexible offset element 574. Regardless of the degree of offset or the rotation of the tube 554, 556, the length of the section of a control cable that passes through the flexible offset element 574 does not change. As a result, the offset assembly does not interfere with the operation of the manipulator 102, proximal joint 104, distal joint 108, or end effector 110.

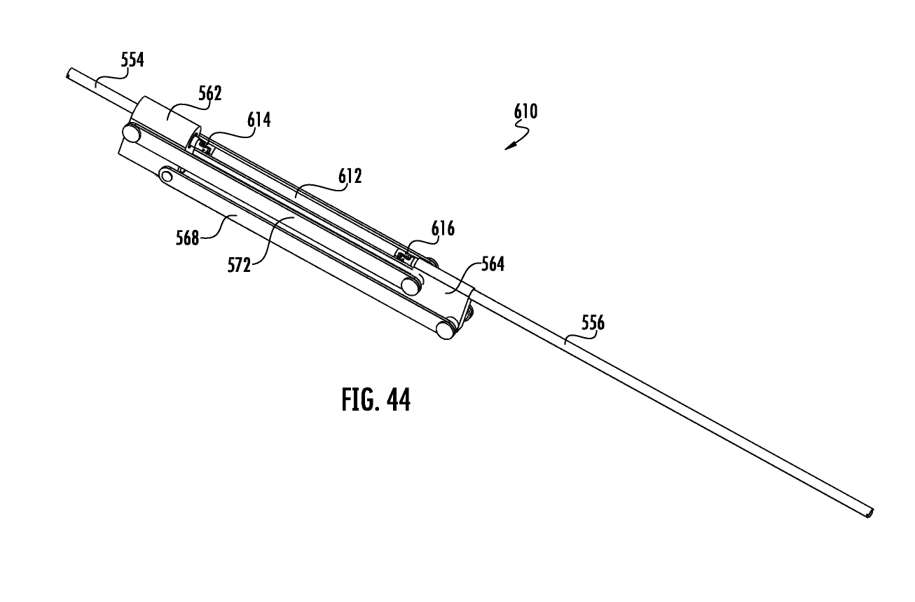

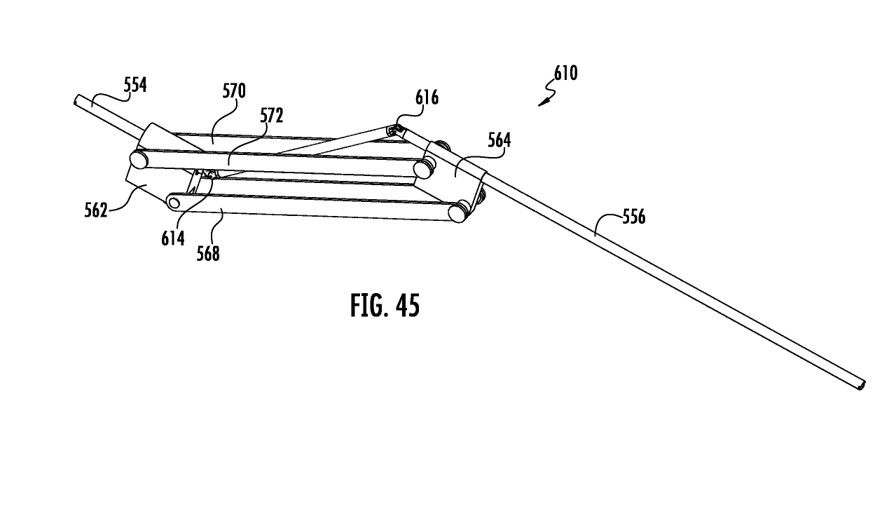

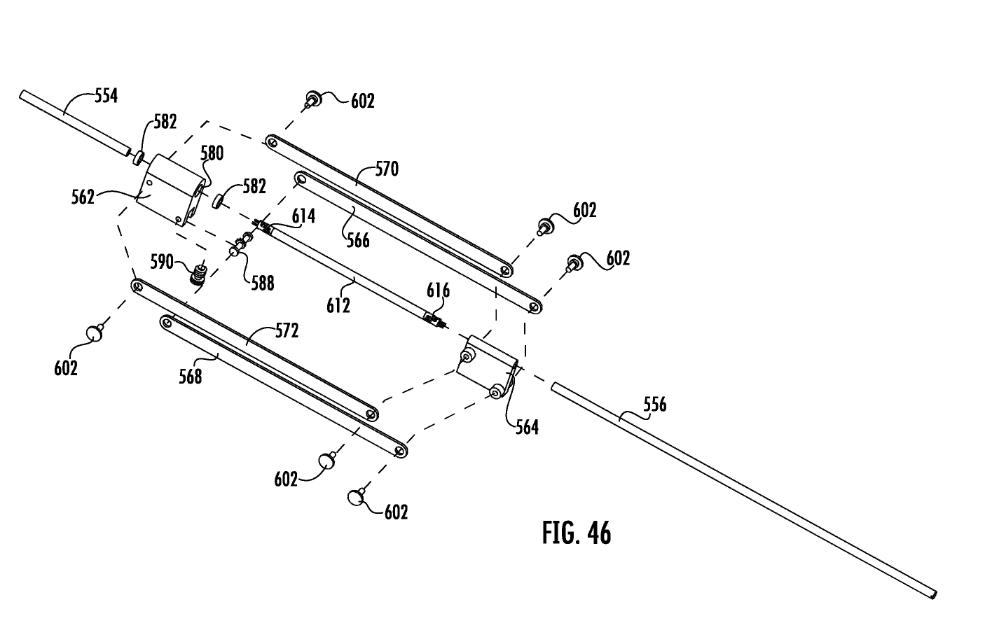

FIGS. 44-46 show another embodiment of a tube offset assembly 610. The flexible offset element 574 has been replaced by a middle tube section 612 with a universal joint 614, 616 at each end connected to the proximal tube 554 and the distal tube, respectively. The construction of these joints 614, 616 is similar to the proximal joint 104 but without the guide pulley 440 (FIG. 27), as is the means by which cables are routed through these joints 614, 616. The deflection of the primary axes of each joint 614, 616 is equal in magnitude and opposite in direction, as is the deflection of their secondary axes. This produces the same effect as the flexible offset element 574, which is that the net length of the section of a cable passing through the offset assembly is unaffected by the degree of offset or rotation of the tube elements 554, 556, 612 in the assembly 610. The presence of the tube offset assembly 610 allows for lateral displacement of the manipulator 102 without interfering with the operation of the instrument.

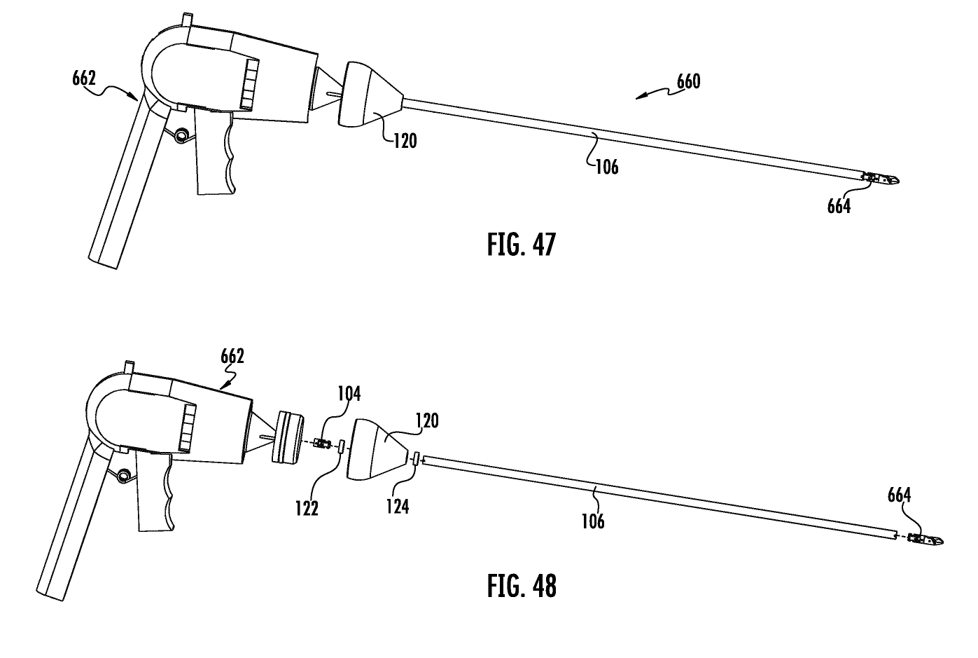

FIGS. 47 and 48 show an embodiment of a surgical instrument 660 incorporating another embodiment of a manipulator 662 and an embodiment of an end segment 664 that includes an integrated distal universal joint and end effector. The end segment 664 is mounted at the distal end of the tube 106. At the proximal end of the tube 106 the manipulator 662 is mounted to the proximal universal joint 104 for controlling the motion of the end segment 664.

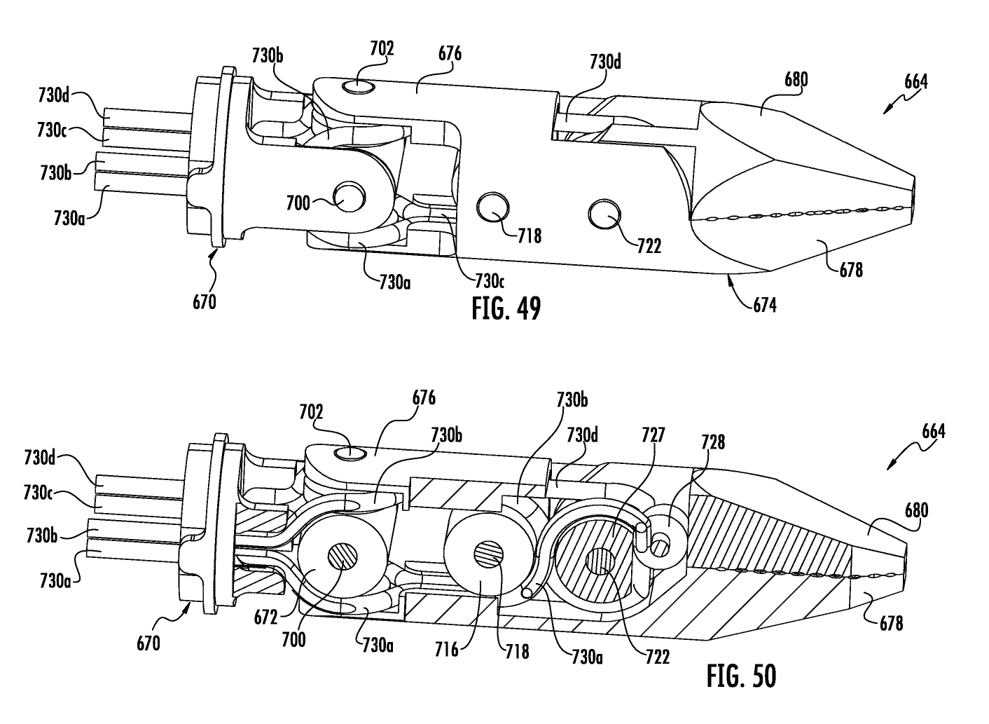

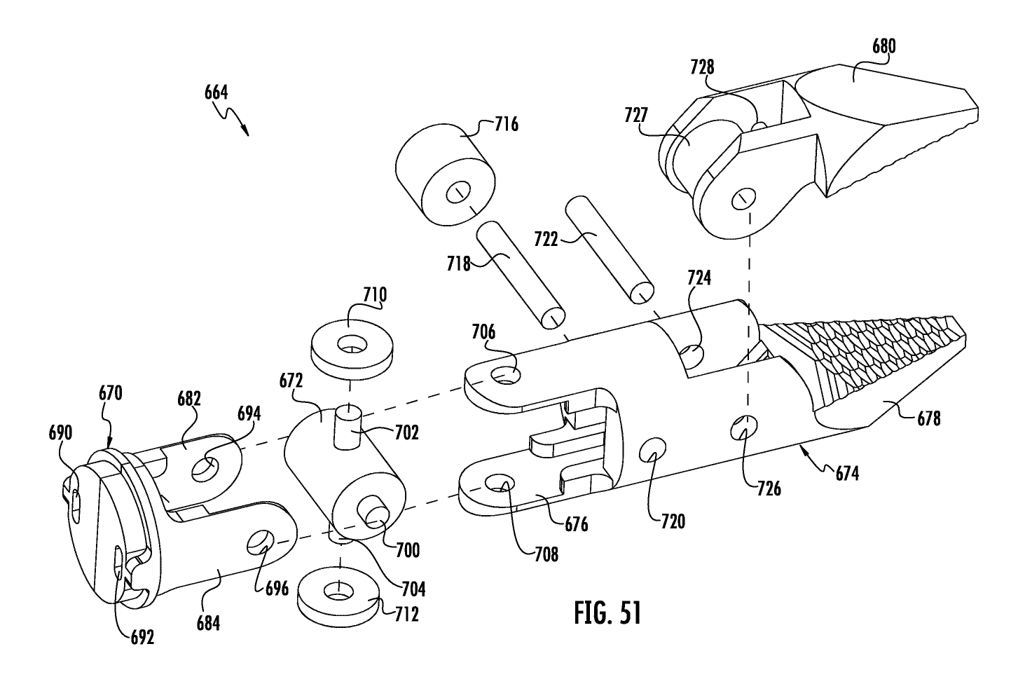

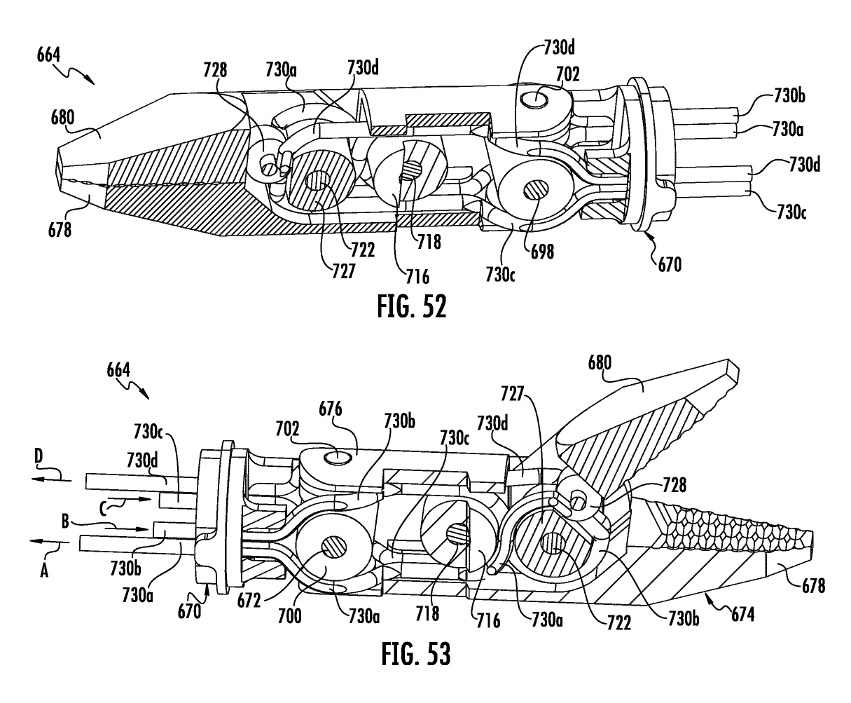

FIGS. 49-55 show the end segment 664, which includes a proximal yoke 670, center block 672, jaw base 674 including a distal yoke portion 676 and a fixed jaw 678, and a pivotally connected jaw 680. The proximal yoke 670 may be made in one part or more than one part 682, 684 as previously described with respect to proximal yoke 138. The proximal yoke 670 likewise defines openings 690, 692 in its proximal end for cables (FIG. 51), and openings 694, 696 in its arms in which lateral, primary joint pins 698, 700 that extend from the center block 672 are mounted.

The primary joint pins 698, 700 define the primary joint axis, and may be one pin that extends through the center block 672. Secondary joint pins 702, 704 that define the secondary joint axis also extend from the center block 672, may be one pin extending through the center block 672, and are received in openings 706, 708 in the arms of the distal yoke portion 676 of the jaw base 674 to connect the jaw base 674 to the center block 672. The primary and secondary axes are substantially perpendicular and intersect, and provide two degrees of freedom for the jaw base 674. Two joint idling pulleys 710, 712 each receive a secondary joint pin 702, 704. In another embodiment of the end segment, the joint idling pulleys 710, 712 may be replaced by round protrusions from either the jaw base 674 or the center block 672. The jaw base 674 houses an idling pulley 716 mounted on a pin 718 that is received in openings 720 (left side opening not visible) in the jaw base 674. The pivotally connected jaw 680 is also mounted on a pin 722 received in openings 724, 726 in the jaw base 674. This jaw 680 includes a pulley feature 727 and a pin feature 728.

There are four control cables 730a, 730b, 730c, 730d that control the motion of the joint and pivotally connected jaw 680. The designations 730a, 730b, 730c, 730d refer to cable lengths, pairs of which 730a, 730b and 730c, 730d may or may not be continuous, but as with the previously described cables 230a, 230b, 230c, 230d, these cables lengths are referred to herein as cables. The pin feature 727 of the pivotally connected jaw 680 is the point at which the cables are distally secured, and may in other embodiments be a swaged component or other mechanism which terminates the cables in a secure manner. None of the control cables move around the pin feature 728.

Cable 730a passes through the proximal yoke 670 and underneath the center block 672, around the bottom joint idling pulley 712 and into the jaw base 674. It then passes under the jaw idling pulley 716 and over the pulley feature 727 of the pivotally connected jaw 680 and connects to the pin feature 728 of the pivotally connected jaw 680.

Cable 730b passes through the proximal yoke 670 and over the center block 672, around the top joint idling pulley 710 and into the jaw base 674. It then passes over the jaw idling pulley 716 and under the pulley feature 727 of the pivotally connected jaw 680 and connects to the pin feature 728 of the pivotally connected jaw 680.

Cable 730c passes through the proximal yoke 670 and underneath the center block 672, around the bottom joint idling pulley 712 and into the jaw base 674. It then passes under the jaw idling pulley 716 and the pulley feature 727 of the pivotally connected jaw 680 and connects to the pin feature 728 of the pivotally connected jaw 680.

Cable 730d passes through the proximal yoke 670 and over the center block 672, around the top joint idling pulley 710 and into the jaw base 674. It then passes over the jaw idling pulley 716 and the pulley feature 727 of the pivotally connected jaw 680 and connects to the pin feature 728 of the pivotally connected jaw 680.

FIG. 53 shows the end segment 664 with the jaw 680 in an open position. This is achieved by retractions A and D of cables 730a and 730d, which relaxes B, D cables 730b and 730c. Retracting cables 730a and 730d, which are diagonally opposed to one another, has no effect on the position of the jaw base 674 relative to either the primary or secondary joint axes. Rather, both cables 730a, 730d exert a torque to open the pivotally connected jaw 680, which subsequently displaces cables 730b and 730c toward the distal end of the end segment 664.

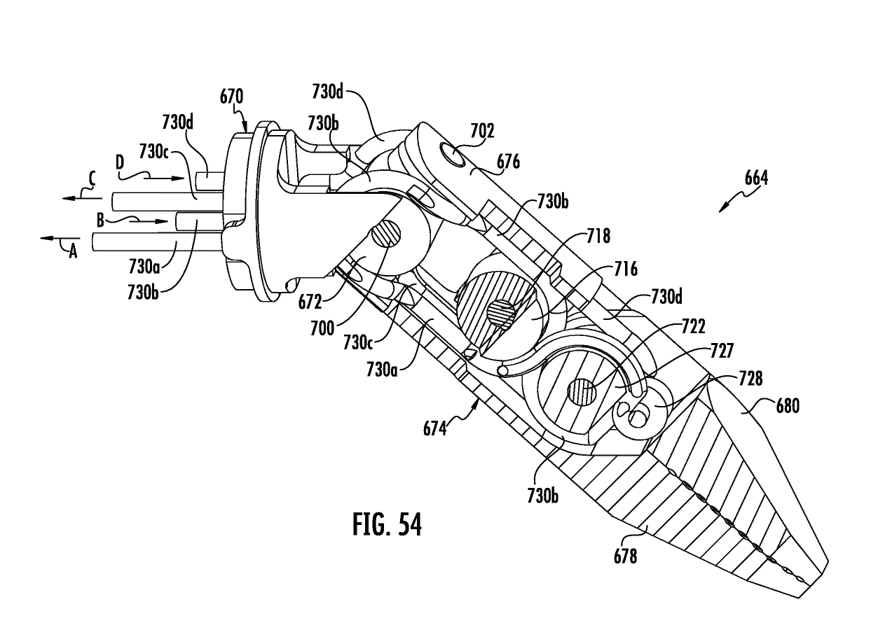

FIG. 54 shows the end segment 664 with cabling such that the jaw base 674 is deflected downward about the primary joint axis. This is achieved by retracting motions A and C for cables 730a and 730c, which relaxes B, D cables 730b and 730d. When cables A and C are retracted, they exert opposite torques on the pivotally connected jaw 680 and thus have no effect on its position relative to the jaw base 674. Instead, the net result is a torque on the center block 672 about the primary joint axis.

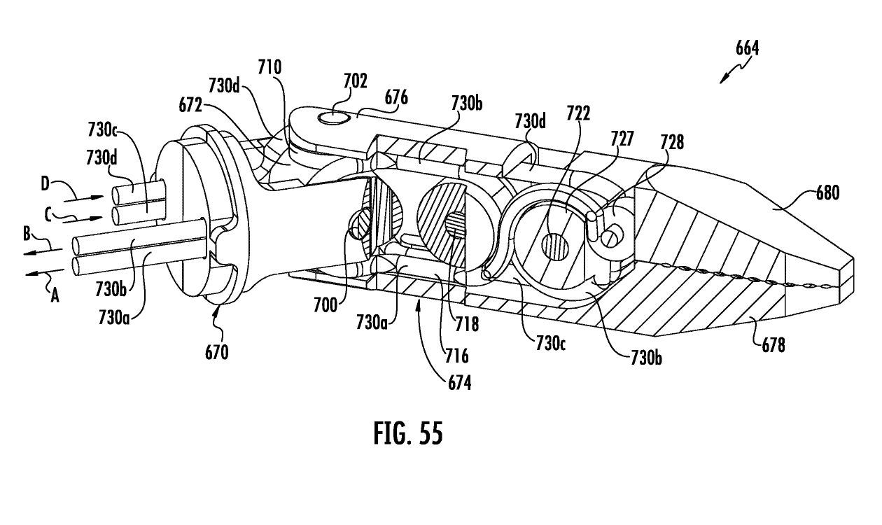

FIG. 55 shows the end segment 664 with cabling such that the jaw base 674 is deflected to the right about the secondary joint axis. This is achieved by retracting motions A and B for cables 730a and 730b, which relaxes C, D cables 730c and 730d. When cables A and B are retracted, they exert opposite torques on the pivotally connected jaw 680 and thus have no effect on its position relative to the jaw base 674. Instead, the net result is a torque on the jaw base 674 about the secondary joint axis.

Since the three motions and their associated control actions are linearly independent, every possible set of cable movements corresponds to a unique and predictable response by the end segment 664, given the cabling is subject to no loss of tension. This provides a simple and effective means of controlling the three degrees of freedom (3DOF) system of the end segment 664 via four control cables 730a, 730b, 730c, 730d, the theoretical minimum.

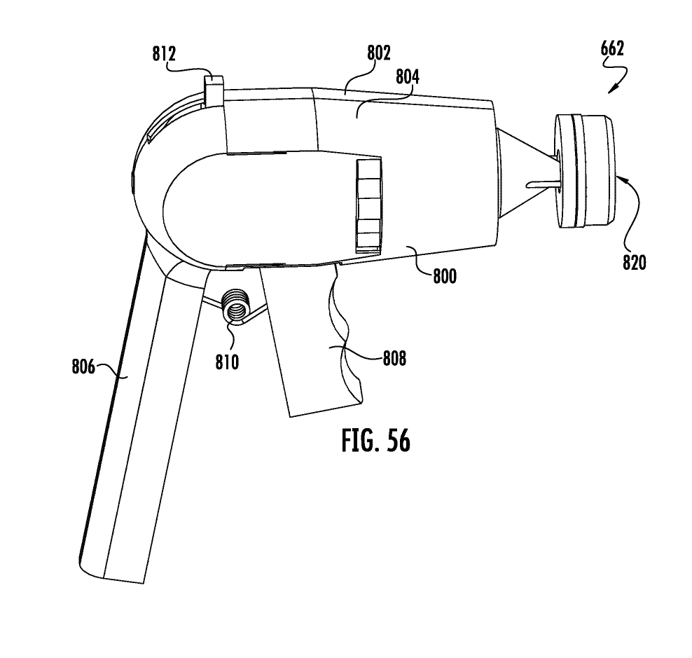

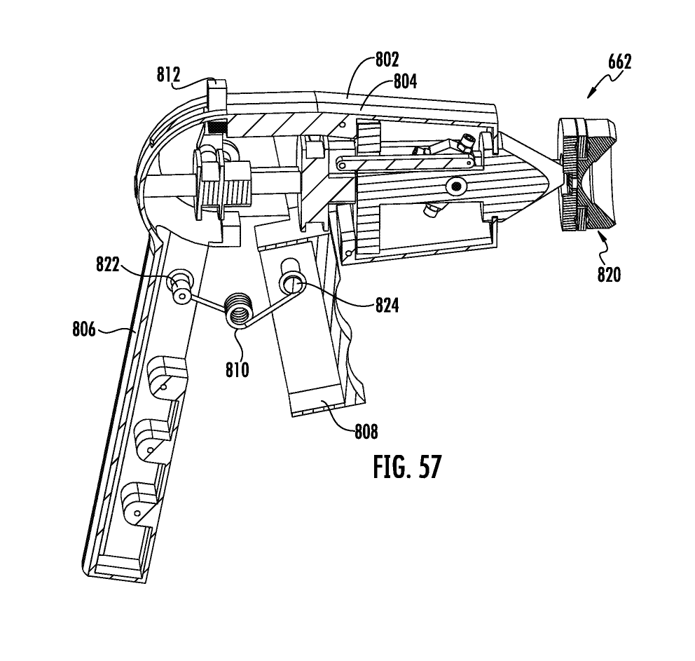

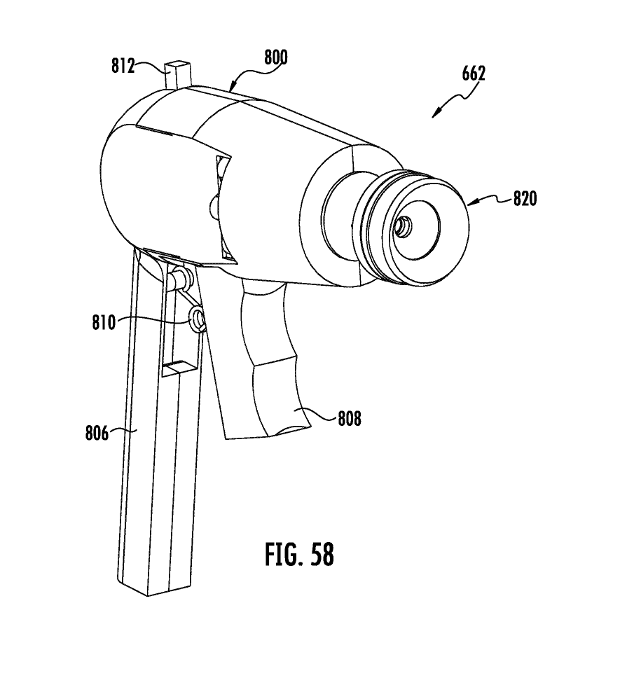

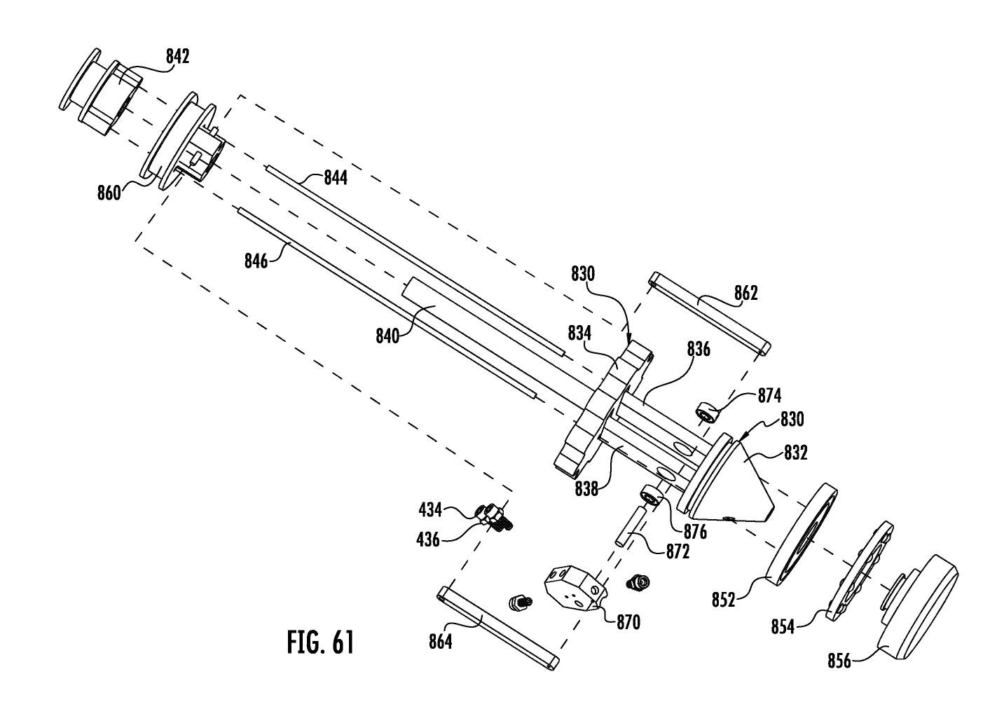

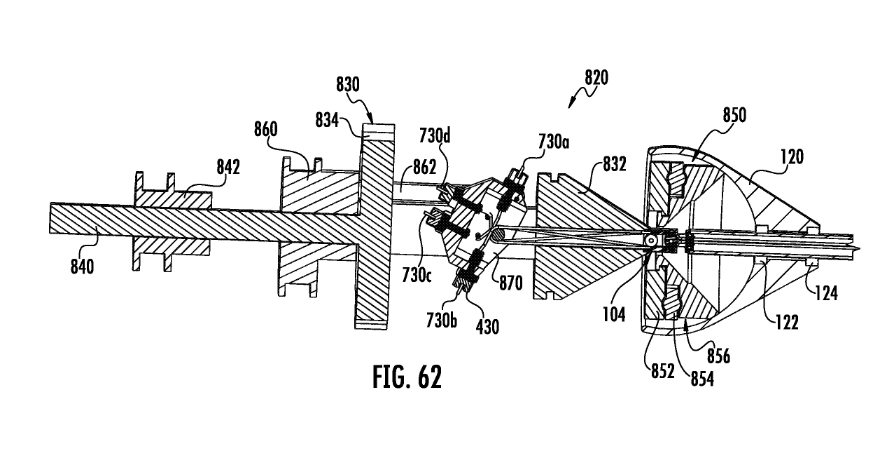

FIGS. 56-71 show the manipulator 662 of the second embodiment of the surgical instrument 660. This embodiment of a manipulator 662 is configured as a pistol-grip handle. As shown in FIGS. 56-59, the manipulator 662 includes a housing 800 that may be made in two parts 802, 804 and include a handle portion 806 adapted to be gripped by a user's hand, a jaw trigger 808 biased with a return spring 810, a brake trigger 812, and control assembly 820 mounted to the housing 800. The jaw trigger 808 controls the opening and closing of the jaws 678, 680 of the end segment 664. The return spring 810 is mounted at one end to a projection 822 in the handle portion 806 of the housing 800 and at the other end to a rod 824 in the jaw trigger 808, and biases the jaw trigger 808 such that the pivotally connected jaw 680 is open when there is no force applied to the jaw trigger 808. The brake trigger 812 locks and unlocks the motion of the proximal joint 104, allowing the user to fix the instrument in any angular position within its range of motion.

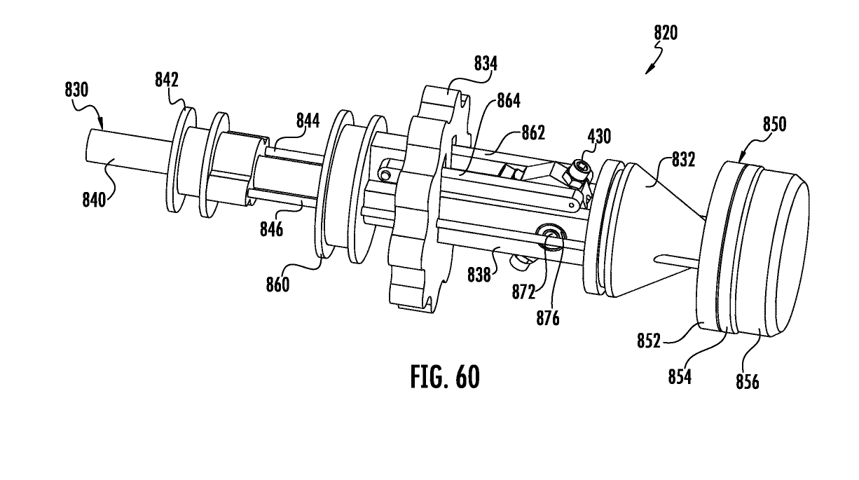

FIGS. 60-62 show the control assembly 820. A chassis 830 has a leading conical face 832 connected to a gear-like flange 834 with parallel, spaced beams 836, 838. A rod portion 840 extends rearward form the flange 834. The flange 834 allows the user to rotate the entire assembly 820 about its longitudinal axis. A brake actuating element 842 slides along the rod portion 840 of the chassis 830. The brake actuating element 842 is connected via two pushrods 844, 846 to the brake assembly 850, which includes a brake collar 852, a brake bearing 854, and a brake 856. Movement of the brake actuating element 842 along the longitudinal axis of the chassis 830 translates directly to a similar movement of the brake assembly 850 due to their rigid connection.

A jaw actuating element 860 also translates linearly along the rod portion 840 of the chassis 830. The jaw actuating element 860 is connected via two actuating links 862, 864 to an anchor 870, which is located between the parallel beams 836, 838. The anchor 870 pivots about a pin 872 received by bearings 874, 876 in openings in the beams 836, 838. The anchor 870 is the proximal point of termination for the four actuating cables that control the end segment 664 and is configured and cabled similarly to the previously described embodiment of an anchor 400 and manipulator 102. Four tensioning assemblies 430 allow the cables to be independently tensioned during assembly such that the position of the proximal joint 104 and distal joint and the jaws of the end segment 664 can be calibrated. Rotation of the anchor 400 in a counterclockwise direction (as viewed from the right side) opens the pivotally connected jaw 680 of the end segment 664. This is accomplished by moving the jaw actuating element 860 toward the rear of the rod portion 840 of the chassis 830.

The proximal universal joint 104 may be the same as previously described, both in design and in cable routing. Alternatively, it may be essentially a mirrored version of the joint in the end segment 664, but without jaws. In addition, pivoting the manipulator 662 has the same effect on the end segment 664 as pivoting the previously described manipulator 102 does on the distal universal joint 108 in the surgical instrument 100, as described with respect to FIG. 17.

As in the previous embodiment of an instrument 100, the joint guard 120 is mounted on two bearings 122, 124. The user can move the manipulator 662 about the proximal joint 104 and lock the instrument 660 at that angular orientation by using the friction between the brake 856 and the joint guard 120. This is achieved by actuating the brake assembly 850 such that the brake 856 is depressed against the inside of the joint guard 120. The joint guard 120 also limits the motion of the manipulator 662 so that the manipulator 662 cannot move beyond the operating range of the proximal joint 104. The conical leading surface 832 of the chassis 830 will hit the joint guard 120 once the manipulator 662 has moved to its limit, preventing further movement. The joint brake bearing 854 and the joint block bearings 122, 124 allow the control assembly 820 to rotate the proximal joint 104 and subsequently the end segment 664 even when the joint 104 is locked in place. This allows free control by the user to rotate the end segment 664 about its longitudinal axis at any time during the operation of the instrument.

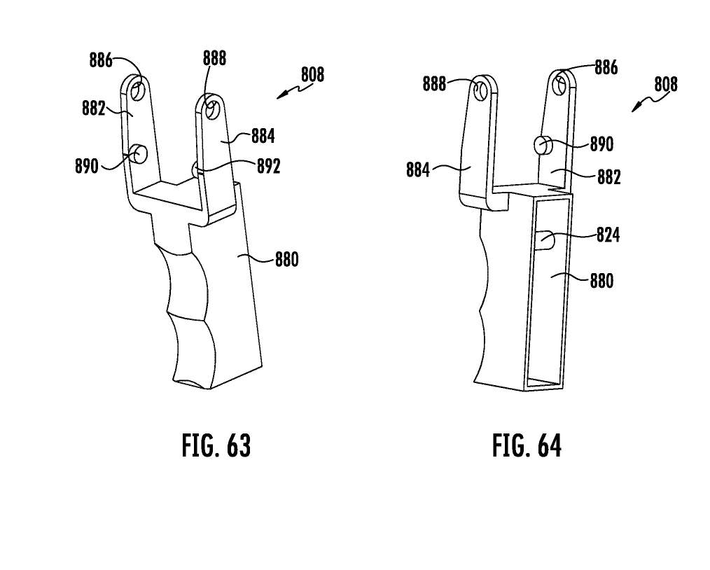

FIGS. 63 and 64 show the jaw trigger 808 of the manipulator 662. The jaw trigger includes a gripping portion 880 and two mounting arms 882, 884. Holes 886, 888 at the free ends of the mounting arms 882, 884 receive protrusions (not shown) on the inside of the housing parts 802, 804. Two round features 890, 892 actuate the jaw actuating element 860 of the control assembly 820. As previously noted, a rod feature 824 receives the return spring 810 that biases the trigger 808 into an open position.

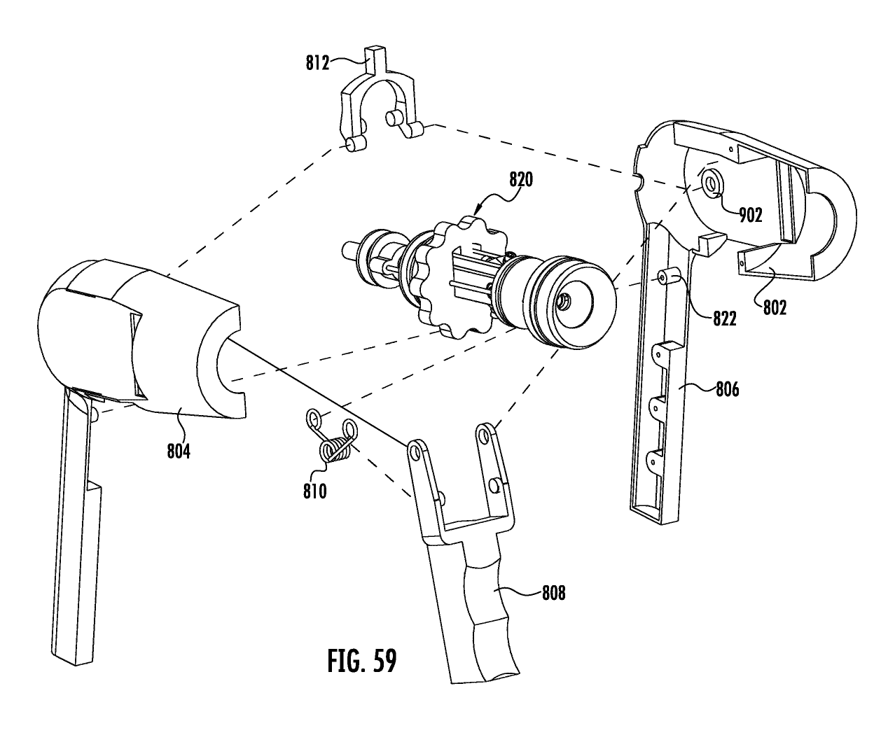

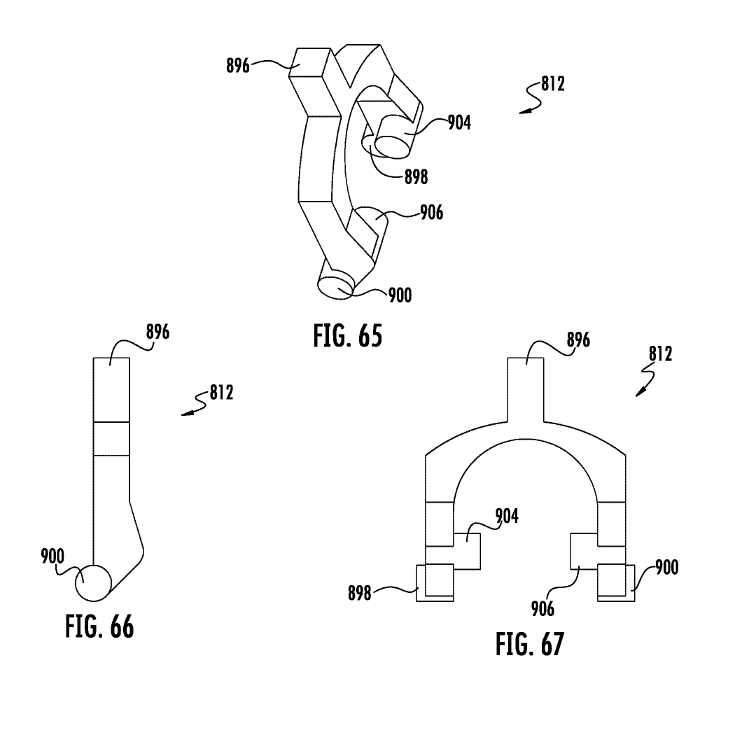

FIGS. 65-67 show the brake trigger 812 of the manipulator 662. A thumb interface feature 896 extends through the housing 800 and allows the user to actuate the brake trigger 812 with their thumb without interrupting other operations of the instrument 660. Two round protrusions 898, 900 that interface with bosses 902 (one visible in FIG. 59) inside the left and right housing parts 802, 804, respectively, define the axis along which the brake trigger 812 pivots. Two round protrusions 904, 906 actuate the brake actuating element 842 of the control assembly 820.

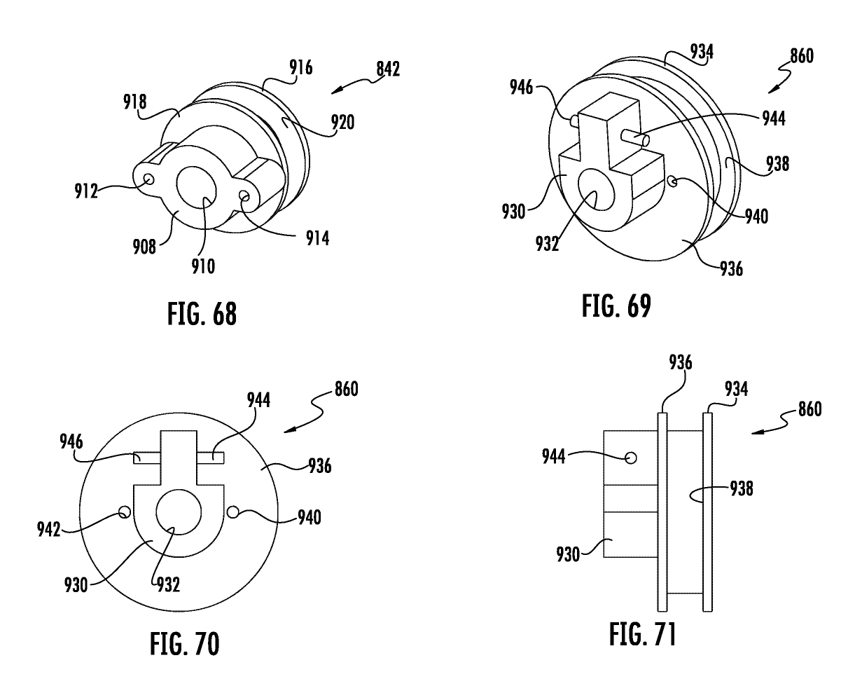

FIG. 68 shows the brake actuating element 842. The brake actuating element 842 includes a body 908 with an opening 910 through which the rod portion 840 of the chassis 830 passes, as well as two recesses 912, 914 that receive and attach to the brake actuating rods 844, 846. Two flanges 916, 918 define a groove 920 that receives protrusions 904, 906 of the brake trigger 812 and allow the brake actuating element 842 to be actuated by longitudinal movement of the protrusions 904, 906 regardless of the angular position of the control assembly 820.

FIGS. 69-71 show the jaw actuating element 860. The jaw actuating element 860 includes a body 930 with an opening 932 through which the rod portion 840 of the chassis 830 passes. Two flanges 934, 936 define a groove 938 that receives round features 890, 892 of the jaw trigger 808 and allow the jaw actuating element 860 to be actuated by longitudinal movement of the round features 890, 892 regardless of the angular position of the control assembly 820. The brake actuating rods 844, 846 pass through two longitudinal openings 940, 942 in the jaw actuating element 860. Transverse pin features 944, 946 provide connections to the actuating links 862, 864 that are connected at the other end to and move the anchor 870.

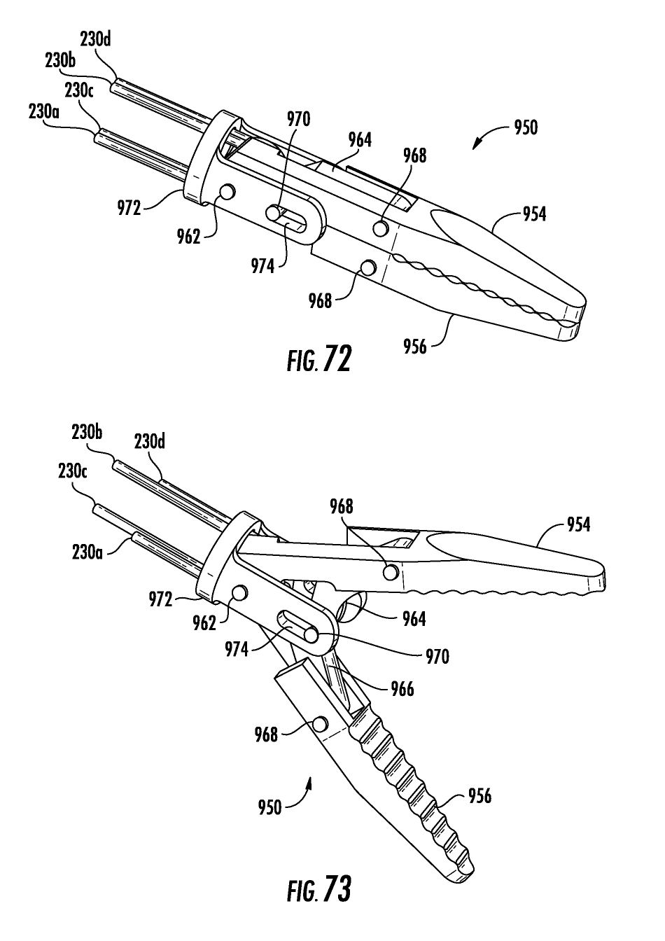

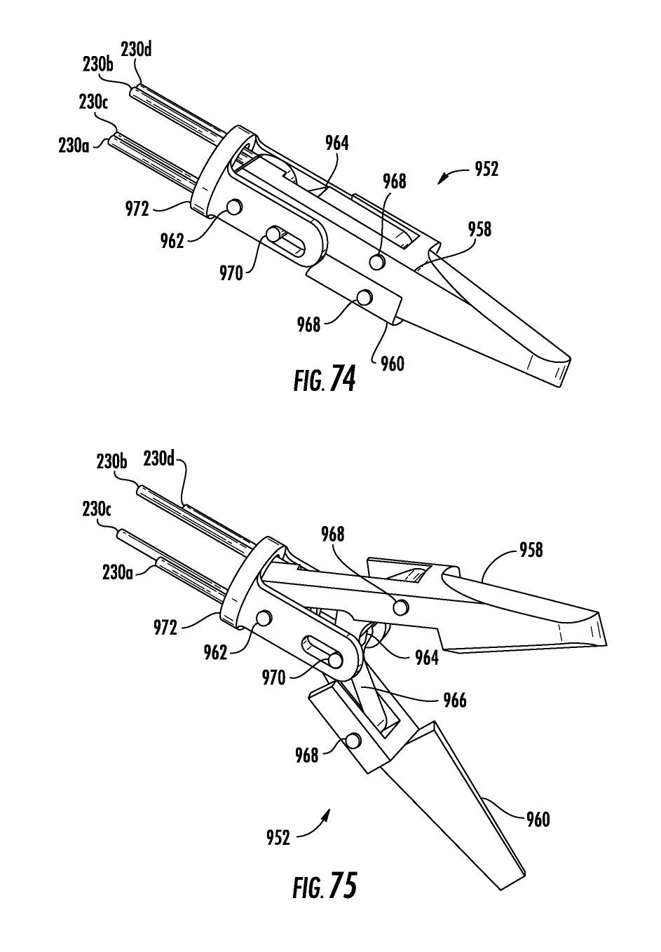

FIGS. 72 and 73 show another embodiment of an end effector 950 including jaws. FIGS. 74 and 75 show another embodiment of an end effector 952 comprising scissor blades forming a scissors apparatus. The jaws and blades are controlled by the pairs of cables 230a, 230b and 230c, 230d in a manner such that the jaws 954, 956 and blades 958, 960, are permitted to rotate in opposite directions but prevented from moving in the same direction. In FIGS. 72 and 73, the cables connect to the jaws 954, 956 which are mounted on a first jaw pin 962 at a proximal end of the jaws 954, 956. The jaws 954, 956 connect to constraining links 964, 966 via pin-like features 968 that may be manufactured as part of the constraining links 964, 966 or may be separate pins that are inserted into the constraining links 964, 966. The pin-like features 968 are inserted into openings intermediate the length of the jaws 954, 956. The constraining links 964, 966 also connect to a sliding pin 970. The jaws 954, 956 and constraining links 964, 966 are mounted to a jaw base 972 with the first jaw pin 962 and the sliding pin 970 extending through openings and slots 974, 976 in the jaw base parts 972, respectively. When the first jaw 954 rotates counterclockwise, the first constraining link 964 rotates counterclockwise and moves in a distal direction, forcing the sliding pin 970 and second constraining link 966 to move distally, which rotates the second jaw 956 clockwise. The configuration of the embodiment of the scissors apparatus 952 (FIGS. 74 and 75) is identical to the end effector 950 shown in FIGS. 72 and 73 except that the jaws 954, 956 are replaced by blades 978, 980. Accordingly, the jaws 954, 956 and blades 978, 980 are constrained to move in opposite directions.

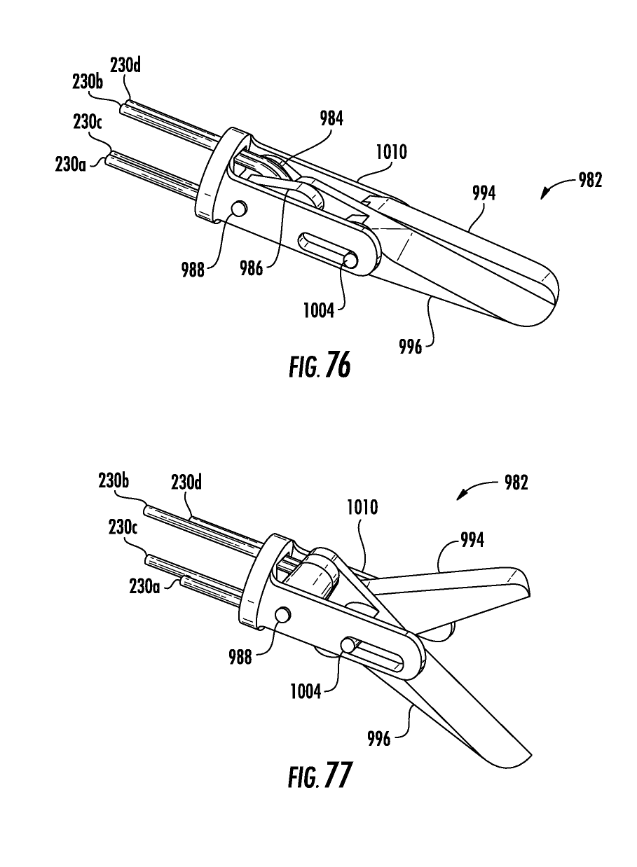

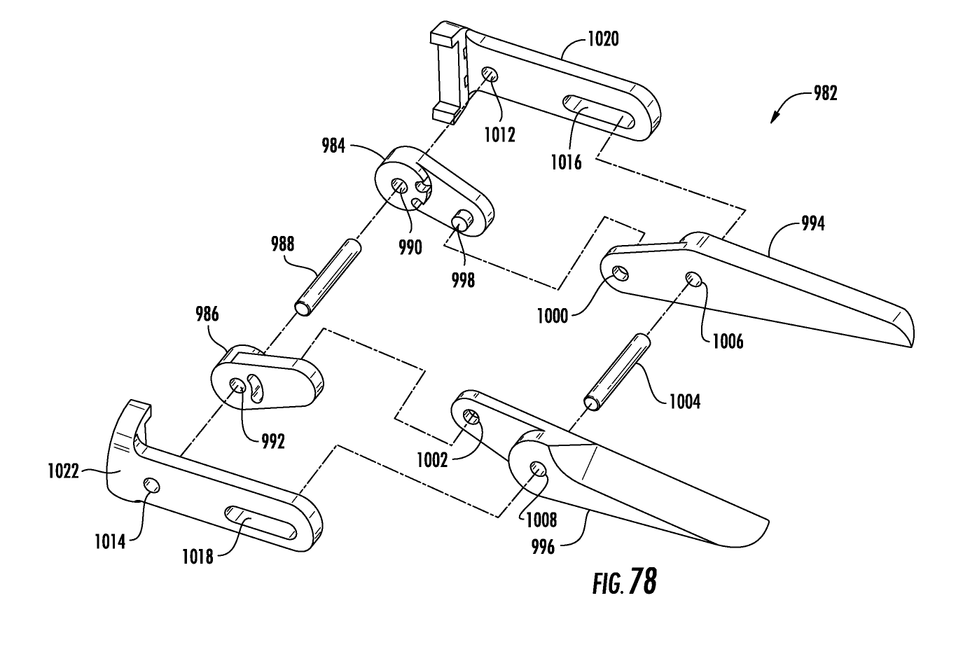

Another embodiment of an end effector 982 is shown in FIGS. 76-78. The end effector 982 is a scissors apparatus and comprises two blades are controlled by pairs of cables 230a, 230b and 230c, 230d in a manner such that the blades are permitted to rotate in opposite directions but prevented from moving in the same direction. In this embodiment of the scissors apparatus 982, cables connect to constraining links 984, 986 which are mounted on a first jaw pin 988 at openings 990, 992 at proximal ends of the constraining links 984, 986. The constraining links 984, 986 connect to blades 994, 996 via pin-like features 998 that may be manufactured as part of the constraining links 984, 986, as shown, or may be separate pins that are inserted into the constraining links 984, 986. The pin-like features 998 are inserted into openings 1000, 1002 at proximal ends of the blades 994, 996. The blades 994, 996 also connect to a sliding pin 1004 at openings 1006, 1008 intermediate the length of the blades. The constraining links 984, 986 and blades 994, 996 are mounted to a jaw base 1010 with the first jaw pin 998 and the sliding pin 1004 extending through openings 1012, 1014 and slots 1016, 1018 in the jaw base parts 1020, 1022, respectively. When the first constraining link 984 rotates counterclockwise, the first blade 994 rotates clockwise and moves in a proximal direction, forcing the sliding pin 1004 and second constraining link 986 to rotate clockwise, which rotates the second blade 996 counterclockwise. Thus, the blades 994, 996 are constrained to move in opposite directions.

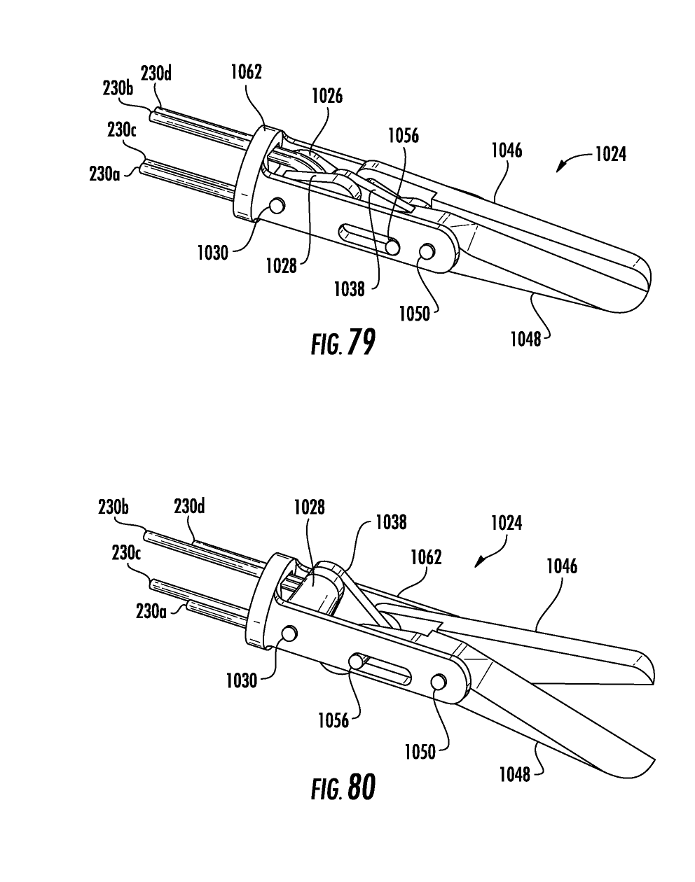

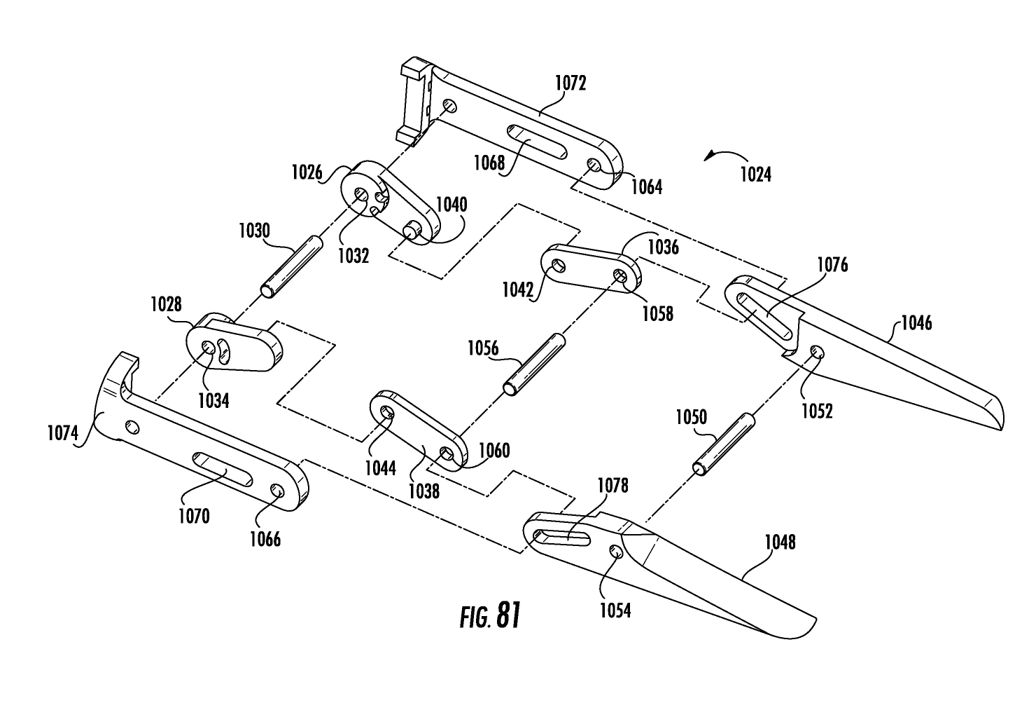

Still another embodiment of an end effector 1024 in which two blades are controlled by pairs of cables 230a, 230b and 230c, 230d in a manner such that the blades are permitted to rotate in opposite directions but prevented from moving in the same direction is shown in FIGS. 79-81. In this embodiment of a scissors apparatus 1024, the cables connect to a first pair of links 1026, 1028 which are mounted on a first jaw pin 1030 at openings 1032, 1034 at proximal ends of the first pair of links 1026, 1028. The first pair of links 1026, 1028 connects to a second pair of links 1036, 1038 via pin-like features 1040 that may be manufactured as part of the links 1026, 1028 as shown or may be separate pins that are inserted into the links 1026, 1028. The pin-like features 1040 are inserted into openings 1042, 1044 at proximal ends of the second pair of links 1036, 1038. The blades 1046, 1048 are mounted on a first jaw pin 1050 at openings 1052, 1054 intermediate the length of the blades 1046, 1048. The blades 1046, 1048 connect to the second pair of links 1036, 1038 via a sliding pin 1056. The second pair of links 1036, 1038 connects to the sliding pin 1056 at openings 1058, 1060 at distal ends of the links 1036, 1038. The blades 1046, 1048 and second pair of links 1036, 1038 are mounted to a jaw base 1062 with the second jaw pin 1050 and the sliding pin 1056 extending through distal openings 1064, 1066 and slots 1068, 1070 in the jaw base parts 1072, 1074 and proximal slots 1076, 1078 the blades 1046, 1048, respectively. When one 1026 of the first pair of links 1026, 1028 rotates counterclockwise, the connected one 1036 of the second pair of links 1036, 1038 rotates clockwise and moves in a proximal direction causing the first blade 1046 to rotate clockwise and move in a proximal direction, forcing the sliding pin 1004 and second 1038 of the second pair of links 1036, 1038 to move proximally, which rotates the second blade 1048 counterclockwise. Thus, the blades 1046, 1048 are constrained to move in opposite directions.

While the materials of the instrument are not intended to be constrained, the material for many of the parts may be expected to be surgical grade, including stainless steel or plastic, or other materials as known to one of ordinary skill in the art. The universal joints, jaw assembly, and tube may be made of stainless steel. The manipulator may be made of hard plastic and metal components. The flexible middle section of the offsetting tube assembly may be made of flexible plastic. Cables may be made of, for example, stainless steel rope, aramid fiber cables, or aligned fiber cables. Other materials may be selected as known to one of ordinary skill in the art. Dimensions may be selected based on the application. Conventional diameters, which may apply to embodiments described herein, include tube, distal universal joint, end effector, and end segment diameters of 5 or 10 mm, or as appropriate for the cannula through which the instrument must pass.

The surgical instrument may include the characteristic of interchangeability of components. For example, the manipulators 102, 662 previously described may be independently provided, may be substituted in place of each other in their respective instruments 100, 660, or may, for example, be incorporated into non-articulating surgical instruments. The distal universal joint 108 and end effector 110 and the end segment 664 may also be substituted in place of each other in their respective instruments 100, 660. Tube offset assemblies may be used independently of the surgical instruments described herein, and may be used with articulated or non-articulated instruments. Further, in some embodiments manually operated manipulators 102, 662 may be replaced by robotic manipulators.

Although only a few exemplary embodiments have been shown and described in considerable detail herein, it should be understood by those skilled in the art that it is not intended to be limited to such embodiments since various modifications, omissions and additions may be made to the disclosed embodiments without materially departing from the novel teachings and advantages, particularly in light of the foregoing teachings. For example, although a manipulator with thumb and index finger actuation is shown or a trigger actuation for the jaw is shown, the novel assembly shown and described herein may be used other types of manipulators and end effectors. Accordingly, we intend to cover all such modifications, omission, additions and equivalents as may be included within the spirit and scope as defined by the following claims. In the claims, means-plus-function clauses are intended to cover the structures described herein as performing the recited function and not only structural equivalents but also equivalent structures. Thus, although a nail and a screw may not be structural equivalents in that a nail employs a cylindrical surface to secure wooden parts together, whereas a screw employs a helical surface, in the environment of fastening wooden parts, a nail and a screw may be equivalent structures.

What is claimed is:

1. An end effector for a surgical instrument having a distal end including an articulation mechanism, the end effector comprising:
a base portion having a longitudinal axis, the base portion defining a linear elongated slot extending along the longitudinal axis, the base portion adapted to be operatively mounted to the articulation mechanism;
a first pair of links, each link of the first pair of links having a first end and a second end, the first ends of the first pair of links pivotally connected to the base portion at a fixed pivot point; and
a second pair of links, each link of the second pair of links having a first end and a second end and pivotally connected at a fixed pivot point to one or the other of the first pair of links, the second pair of links pivotally connected together through the slot of the base portion for linear movement along the slot relative to the base portion,
wherein the linear movement of the pivotally connected second pair of links relative to the base portion constrains the movement of the first pair of links to relative rotation only in opposite directions.

2. An end effector as recited in claim 1, wherein the fixed pivot point of the base portion is spaced in a first direction along the longitudinal axis of the base portion from the slot, and the fixed pivot point of the first pair of links is intermediate along the length of the each of the first pair of links.

3. An end effector as recited in claim 1, wherein the fixed pivot point of the base portion is spaced in a first direction along the longitudinal axis of the base portion from the slot, and the fixed pivot point of the first pair of links is at the second end of each of the first pair of links.

4. An end effector as recited in claim 1, wherein the fixed pivot point of the base portion is spaced in a second direction along the longitudinal axis of the base portion from the slot, and the fixed pivot point of the first pair of links is at the first end of each of the first pair of links.

5. An end effector as recited in claim 2, further comprising a third pair of links having a first end and a second end and pivotally connected together at a fixed point, each of the third pair of links defining an elongated slot spaced toward the first end from the fixed pivotal connection, the third pair of links pivotally connected together through the slot of the base portion and the slots of the third pair of links to the second pair of links for linear movement along the slots relative to the base portion, wherein the third pair of links is configured for relative rotation in only one direction.

6. A surgical instrument comprising:
an articulation mechanism;
an end effector including
a base portion having a longitudinal axis, the base portion defining a linear elongated slot extending along the longitudinal axis, the base portion configured to be operatively mounted to the articulation mechanism,
a first pair of links, each link of the first pair of links having a first end and a second end, the first ends of the first pair of links pivotally connected to the base portion at a fixed pivot point; and
a second pair of links, each link of the second pair of links having a first end and a second end and pivotally connected at a fixed pivot point to one or the other of the first pair of links, the second pair of links pivotally connected together through the slot of the base portion for linear movement along the slot relative to the base portion,
wherein the second pair of links is configured for concurrent relative rotation only in opposite directions.

7. A surgical instrument as recited in claim 6, wherein the fixed pivot point of the base portion is spaced in a first direction along the longitudinal axis of the base portion from the slot, and the fixed pivot point of the first pair of links is intermediate along the length of the each of the first pair of links.

8. A surgical instrument as recited in claim 6, wherein the fixed pivot point of the base portion is spaced in a first direction along the longitudinal axis of the base portion from the slot, and the fixed pivot point of the first pair of links is at the second end of each of the first pair of links.

9. A surgical instrument as recited in claim 6, wherein the fixed pivot point of the base portion is spaced in a second direction along the longitudinal axis of the base portion from the slot, and the fixed pivot point of the first pair of links is at the first end of each of the first pair of links.

10. A surgical instrument as recited in claim 7, further comprising a third pair of links having a first end and a second end and pivotally connected together at a fixed point, each of the third pair of links defining an elongated slot spaced toward the first end from the fixed pivotal connection, the third pair of links pivotally connected together through the slot of the base portion and the slots of the third pair of links to the second pair of links for linear movement along the slots relative to the base portion, wherein the third pair of links is configured for relative rotation in only one direction.

11. A surgical instrument as recited in claim 6, wherein the articulation mechanism comprises four movable cables, wherein two cables control rotation of one of the first pair of links, and the other two cables control the rotation of the other of the first pair of links for relative rotation of the first pair of drive links.

12. A surgical instrument as recited in claim 6, wherein the articulation mechanism comprises means for pivoting about two axes of rotation.

13. A surgical instrument as recited in claim 12, wherein the two axes of rotation are substantially perpendicular.

14. A surgical instrument as recited in claim 12, wherein each axes of rotation passes through a pin joint.

15. A surgical instrument as recited in claim 6, wherein the articulation mechanism comprises means for pivoting about two axes of rotation, and four movable cables, wherein two of the cables control rotation about one of the two axes of rotation.

16. A surgical instrument as recited in claim 15, wherein two of the cables control rotation about the other of the two axes of rotation.

17. A surgical instrument as recited in claim 6, wherein the articulation mechanism comprises four movable cables for causing movement of the first and second pair of links.
